SEQUENCE LISTING (22 sequences)

SEQ ID NO: 1 - protein, 9 aa, Homo sapiens
EIDESLIFY

SEQ ID NO: 2 - protein, 9 aa, Human immunodeficiency virus
SLYNTVATL

SEQ ID NO: 3 - protein, 10 aa, Human immunodeficiency virus
QVPLRPMTYK

SEQ ID NO: 4 - protein, 9 aa, Homo sapiens
AFLPWHRLF

SEQ ID NO: 5 - protein, 9 aa, Homo sapiens
LLALLMAGL

SEQ ID NO: 6 - protein, 9 aa, Homo sapiens
ALQPGTALL

SEQ ID NO: 7 - protein, 9 aa, Homo sapiens
ALLPALGLL

SEQ ID NO: 8 - protein, 9 aa, Homo sapiens
ALLMAGLAL

SEQ ID NO: 9 - protein, 9 aa, Homo sapiens
ALQPAAAIL

SEQ ID NO: 10 - protein, 9 aa, Homo sapiens
LLPALGLLL

SEQ ID NO: 11 - protein, 9 aa, Homo sapiens
QLGEQCWTA

SEQ ID NO: 12 - protein, 9 aa, Homo sapiens
ALLCYSCKA

SEQ ID NO: 13 - protein, 9 aa, Homo sapiens
AILALLPAL

SEQ ID NO: 14 - protein, 9 aa, Homo sapiens
RIRAVGLLT

SEQ ID NO: 15 - protein, 9 aa, Homo sapiens
DYYVGKKNI

SEQ ID NO: 16 - protein, 9 aa, Homo sapiens
YYVGKKNIT

SEQ ID NO: 17 - protein, 9 aa, Influenza virus
GILGFVFTL

SEQ ID NO: 18 - protein, 9 aa, Influenza virus
ILRGSVAHK

SEQ ID NO: 19 - protein, 9 aa, Epstein-barr virus
RYEDPDAPL

SEQ ID NO: 20 - DNA, 990 bp, Homo sapiens
gtgaccacgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctaggctctggggggccccgctgcagcccacactgggtgtggtgccccaggcctctgtgccactcctcacacacccggcccagtgggagcctgtcctggttcctgaggcacatcctaacgcaagtctgaccatgtatgtctgcgcccctgtcccccaccctgaccctcccatggccctctccaggactcccacccggcagatcggctctattgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccggtaaaggctgagatgaagtggactgagtagaactggaggacaggagtcgacgtgagttcctgggagtctccagagatggggcctggaggcctggaggaaggggccaggcctcacattcgtggggctccctgaatggcagcctcagcacagcgtaggcccttaataaacacctgttggataaaaaaaaaaaaaaaaaaaaaaaaa

SEQ ID NO: 21 - DNA, 1025 bp, Homo sapiens
gtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgccgccatccttgcgctgctccctgcactcggcctgctgctctggggacccggccagctataggctctggggggccccgctgcagcccacactgggtgtggtgccccaggcctctgtgccactcctcacagacctggcccagtgggagcctgtcctggttcctgaggcacatcctaacgcaagtctgaccatgtatgtctgcacccctgtcccccaccctgaccctcccatggccctctccaggactcccacccggcagatcagctctagtgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccagtaaaggctgagatgaagtggactgagtagaactggaggacaagagtcgacgtgagttcctgggagtctccagagatggggcctggaggcctggaggaaggggccaggcctcacattcgtggggctccctgaatggcagcctgagcacagcgtaggcccttaataaacacctgttggataagcaaaaaaaaaaaaaaaaaaaaaaaaaaaaaaaaaaaaaaaaaaaaaaaaaaaaaaaa

SEQ ID NO: 22 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

(19) United States
(12) Patent Application Publication
(10) Pub. No.: US 2007/0059315 A1
(43) Pub. Date: Mar. 15, 2007

(54) Prostate stem cell antigen vaccines and uses thereof

(76) Inventor: Elizabeth M. Jaffee, Lutherville, MD (US)

Correspondence Address: MORRISON & FOERSTER LLP, 755 PAGE MILL RD, PALO ALTO, CA, 94304-1018, US

(21) Appl. No.: 11/489,762

(22) Filed: Jul. 19, 2006

Related U.S. Application Data
(63) Continuation-in-part of application No. PCT/US06/01424, filed on Jan. 13, 2006.
(60) Provisional application No. 60/643,703, filed on Jan. 13, 2005.

(51) Int. Cl. A61K 39/395; A61K 39/00

(52) U.S. Cl. 424/155.1; 424/185.1

(57) ABSTRACT

This invention relates to the identification of prostate stem cell antigen (PSCA) as a target of clinically relevant antitumor immune responses. The invention provides vaccines comprising PSCA, or fragments thereof, which are useful in inducing antitumor immune responses, including PSCA specific CD8+ T cell responses. Methods of using the compositions to treat cancer are also provided. The invention further provides methods of identifying compounds useful in antitumor vaccines and methods of assessing the responses of patients to cancer immunotherapy.

### CROSS REFERENCE TO RELATED APPLICATIONS

This application is a continuation-in-part of International Application No. PCT/US2006/001424, filed Jan. 13, 2006, which claims the priority benefit of U.S. Provisional Application Ser. No. 60/643,703, filed Jan. 13, 2005, each of which is hereby incorporated by reference herein in its entirety.

### STATEMENT REGARDING FEDERALLY SPONSORED RESEARCH OR DEVELOPMENT

This invention was made, in part, with government support under NIH/NCI Grant No. R01CA95012 and NIH/NCI Grant No. P50CA62924. The government may have certain rights in the invention.

### FIELD OF THE INVENTION

This invention relates to the field of cancer therapeutics and prognosis. More specifically, in some aspects, this invention relates to the identification of PSCA as a tumor antigen against which clinically relevant anti-cancer immune responses can be induced, as well as use of PSCA, or fragments thereof, in cancer vaccines or for cancer prognosis.

### BACKGROUND OF THE INVENTION

An important component to the design and evaluation of cancer immunotherapies is the identification of specific tumor antigens that are the targets of clinically relevant anti-tumor responses. There is a tremendous ongoing effort to design “antigen-specific” immunotherapy in which specific tumor antigens are incorporated into peptide, protein, recombinant DNA, recombinant virus, recombinant bacteria, or recombinant yeast-based vaccines. These vaccines are utilized-to immunize patients with cancers expressing the relevant tumor-specific antigen. Alternative approaches to cancer immunotherapy include the adoptive transfer of monoclonal antibodies or T cells specific for tumor antigens. Preliminary clinical results in trials of vaccines, T cell adoptive transfer and monoclonal antibody transfer, validate the utility of these approaches in anti-cancer therapy.

For every cancer immunotherapy approach, it is desirable to determine which among the many candidate tumor specific or tumor selective antigens are the best targets for generation of immune responses. Molecular genetics has demonstrated that tumors express both unique antigens—generated by mutational events in their genome—and tumor selective antigens that represent over-expressed proteins due to altered transcription levels of their genes or altered post-transcriptional processing resulting in increased stability of the protein. In addition, tissue specific antigens that are also expressed by tumors arising from that tissue type may also represent potential targets for immunotherapy, particularly when the tumor is derived from a tissue dispensable to life (such as prostate cancer, breast cancer, melanoma, pancreatic cancer, ovarian cancer). While each of these categories of antigens provides a large number of potential antigenic targets, it is now clear that there are many processes that dramatically narrow the subset of antigens actually recognized by the immune system. For T cell recognition of tumors, peptides derived from a candidate antigen must be effectively presented by MHC molecules of both the tumor and host in order to be relevant targets. In addition, constraints on the T cell repertoire, as well as mechanisms of immune tolerance, further restrict the number of antigens against which effective immune responses can be generated. One of the central goals in cancer immunotherapy has therefore been to identify the antigens against which clinically relevant immune responses can be elicited.

Previous approaches to identify immunologically relevant antigens have involved the generation of tumor specific T cell lines and clones followed by the application of these T cell lines and clones to screen cDNA libraries for the recognized tumor antigen. This approach has identified candidate antigens in melanoma as well as a number of other cancers. However, the process of long term culture of these T cells has the possibility of selecting for antigenic specificities which are not representative of the immunodominant antigenic targets in vivo. In addition, most of the antigens identified by this approach utilize T cell lines and clones cultured from patients with actively growing cancer and therefore, do not necessarily correlate with clinically effective anti-cancer immune responses.

### BRIEF SUMMARY OF THE INVENTION

Prostate stem cell antigen (PSCA) is a protein that is overexpressed in a large proportion of pancreatic cancers and other cancers as well, including prostate cancer. The present invention relates in part to the identification of PSCA as a relevant tumor antigen capable of being recognized by T cells from pancreatic cancer patients who have responded to immunotherapy. These results establish this antigen as a marker for immune responses in patients with tumors that overexpress PSCA (e.g., pancreatic cancer and prostate cancer) who are receiving active immunotherapy (vaccines) and adoptive immunotherapy (transfer of T cells and/or antibodies) of cancer. These results also establish this antigen as a clinically effective target for cancer immunotherapy. Accordingly, in some aspects, the invention provides a variety of compositions useful as cancer vaccines, methods of using those compositions to treat cancer in mammals (e.g., humans), methods of assessing whether a patient is having a favorable response to a cancer vaccine, and methods of screening compositions as candidates for vaccines.

In one aspect, the invention provides a method of inducing a T-cell response to a tumor that expresses prostate stem cell antigen (PSCA), said method comprising administering to a mammal who has said tumor or who has had said tumor removed, a composition comprising a polypeptide comprising an MHC class I-binding epitope and/or an MHC class II-binding epitope, whereby a T-cell response to PSCA is induced in the mammal. In some embodiments, the composition does not comprise a whole tumor cell. In some embodiments, the polypeptide comprises an MHC class I-binding epitope of PSCA. In some embodiments, the polypeptide comprises an MHC class II-binding epitope of PSCA. In some embodiments, the polypeptide comprises PSCA. In some other embodiments, the polypeptide comprises a fragment of PSCA.

In another aspect, the invention provides a method of inducing a T-cell response to a tumor that expresses prostate stem cell antigen (PSCA), said method comprising administering to a mammal who has said tumor or who has had said tumor removed, a composition comprising a polynucleotide encoding a polypeptide comprising an MHC class I-binding epitope and/or an MHC class II-binding epitope, whereby a T-cell response to PSCA is induced in the mammal. In some embodiments, the composition does not comprise a whole tumor cell. In some embodiments, the polypeptide comprises an MHC class I-binding epitope of PSCA. In some embodiments, the polypeptide comprises an MHC class II-binding epitope of PSCA. In some embodiments, the polypeptide comprises PSCA. In some other embodiments, the polypeptide comprises a fragment of PSCA.

In another aspect, the invention provides a method of treating cancer in a mammal, comprising: administering to the mammal a composition comprising a polypeptide comprising an MHC class I-binding epitope and/or an MHC class II-binding epitope, whereby a T-cell response to PSCA is induced in the mammal. In some embodiments, the mammal has a PSCA-expressing tumor or has had a PSCA-expressing tumor removed. In some embodiments, the composition does not comprise a whole tumor cell. In some embodiments, the polypeptide comprises an MHC class I-binding epitope of PSCA. In some embodiments, the polypeptide comprises an MHC class II-binding epitope of PSCA. In some embodiments, the tumor-expressing tumor overexpresses PSCA relative to the normal tissue from which the tumor is derived. In some embodiments, the cancer is pancreatic cancer, bladder cancer, or prostate cancer. In some embodiments, the polypeptide comprises PSCA. In some other embodiments, the polypeptide comprises a fragment of PSCA.

In another aspect, the invention provides a method of treating cancer in a mammal who has a PSCA-expressing tumor or who has had a PSCA-expressing tumor removed, comprising: administering to the mammal a composition comprising a polypeptide comprising an MHC class I-binding epitope and/or an MHC class II-binding epitope, whereby a T-cell response to PSCA is induced in the mammal; and further treating the mammal with chemotherapy, radiation, surgery, hormone therapy, or additional immunotherapy. In some embodiments, the polypeptide comprises an MHC class I-binding epitope of PSCA. In some embodiments, the polypeptide comprises an MHC class II-binding epitope of PSCA. In some embodiments, the composition does not comprise a whole tumor cell.

In still another aspect, the invention provides a method of treating cancer in a mammal, comprising: administering to the mammal a composition comprising a polynucleotide encoding a polypeptide comprising an MHC class I-binding epitope and/or an MHC class II-binding epitope, whereby a T-cell response to PSCA is induced in the mammal. In some embodiments, the mammal has a PSCA-expressing tumor or has had a PSCA-expressing tumor removed. In some embodiments, the composition does not comprise a whole tumor cell. In some embodiments, the polypeptide comprises an MHC class I-binding epitope of PSCA. In some embodiments, the polypeptide comprises an MHC class II-binding epitope of PSCA. In some embodiments, the tumor-expressing tumor overexpresses PSCA relative to the normal tissue from which the tumor is derived. In some embodiments, the cancer is pancreatic cancer, bladder cancer, or prostate cancer. In some embodiments, the polypeptide comprises PSCA. In some other embodiments, the polypeptide comprises a fragment of PSCA.

In another aspect, the invention provides a method of treating cancer in a mammal who has a PSCA-expressing tumor or who has had a PSCA-expressing tumor removed, comprising: administering to the mammal a composition comprising a polynucleotide encoding a polypeptide comprising an MHC class I-binding epitope and/or an MHC class II-binding epitope, whereby a T-cell response to PSCA is induced in the mammal; and further treating the mammal with chemotherapy, radiation, surgery, hormone therapy, or additional immunotherapy. In some embodiments, the polypeptide comprises an MHC class I-binding epitope of PSCA. In some embodiments, the polypeptide comprises an MHC class II-binding epitope of PSCA. In some embodiments, the composition does not comprise a whole tumor cell.

In still another aspect, the invention provides a vaccine that induces a T cell response to a PSCA-expressing tumor cell in a human, comprising: a polypeptide comprising an MHC class I-binding epitope and/or an MHC class II-binding epitope. In some embodiments, the vaccine further comprises an adjuvant. In some embodiments, the vaccine does not comprise a whole tumor cell. In some embodiments, the polypeptide comprises an MHC class I-binding epitope of PSCA. In some embodiments, the polypeptide comprises an MHC class II-binding epitope of PSCA.

In still another aspect, the invention provides a vaccine that induces a T cell response to a PSCA-expressing tumor cell in a human, comprising: a polynucleotide encoding a polypeptide comprising an MHC class I-binding epitope and/or an MHC class II-binding epitope. In some embodiments, the vaccine further comprises an adjuvant. In some embodiments, the vaccine does not comprise a whole tumor cell. In some embodiments, the polypeptide comprises an MHC class I-binding epitope of PSCA. In some embodiments, the polypeptide comprises an MHC class II-binding epitope of PSCA.

In a further aspect, the invention provides a vaccine that induces a T cell response to PSCA-expressing tumor cell in a human, comprising: a whole cell from a tumor cell line that has been selected or modified to overexpress a polypeptide relative to the tumor cell line prior to selection or modification, wherein the polypeptide comprises an MHC class I-binding epitope and/or an MHC class II-binding epitope; and an adjuvant. In some embodiments, the polypeptide comprises an MHC class I-binding epitope of PSCA. In some embodiments, the polypeptide comprises an MHC class II-binding epitope of PSCA. In some embodiments, the vaccine comprises a recombinant vector comprising a bacterium, virus or yeast expressing the polypeptide. In some embodiments, the polypeptide further comprises an MHC class II-binding epitope. In some embodiments, the polypeptide comprises a plurality of MHC class I-binding epitopes of PSCA. In some embodiments, the polypeptide comprises a plurality of MHC class I-binding epitopes of PSCA that bind the allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the T-cell response comprises induction of PSCA specific CD8+ T cells. In some embodiments, the vaccine further comprises an adjuvant or a non-PSCA antigen. In some embodiments, the polypeptide does not comprise PSCA, but rather comprises a fragment of PSCA, wherein the fragment comprises the MHC class I epitope. In some embodiments, the fragment is at least 8 amino acids in length.

In another aspect, the invention provides a method of inducing a T-cell response to a tumor that expresses prostate stem cell antigen (PSCA), said method comprising administering to a mammal who has said tumor or who has had said tumor removed, a composition comprising a whole cell from a tumor cell line that has been selected or modified to overexpress a polypeptide relative to the tumor cell line prior to selection or modification, wherein the polypeptide comprises an MHC class I-binding epitope and/or an MHC class II-binding epitope, whereby a T-cell response to PSCA is induced in the mammal. In some embodiments, the polypeptide comprises an MHC class I-binding epitope of PSCA. In some embodiments, the polypeptide comprises an MHC class II-binding epitope of PSCA. In some embodiments, the composition comprises a recombinant vector comprising a bacterium, virus or yeast expressing the polypeptide. In some embodiments, the polypeptide further comprises an MHC class II-binding epitope. In some embodiments, the polypeptide comprises a plurality of MHC class I-binding epitopes of PSCA. In some embodiments, the polypeptide comprises a plurality of MHC class I-binding epitopes of PSCA that bind the allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the T-cell response comprises induction of PSCA specific CD8+ T cells. In some embodiments, the composition further comprises an adjuvant or a non-PSCA antigen. In some embodiments, the polypeptide does not comprise PSCA, but rather comprises a fragment of PSCA, wherein the fragment comprises the MHC class I epitope. In some embodiments, the fragment is at least 8 amino acids in length. In some embodiments the tumor is a tumor that overexpresses prostate stem cell antigen relative to the normal tissue from which the tumor is derived. For example, in some embodiments, the tumor is a pancreatic cancer, a bladder cancer, or a prostate cancer. In some embodiment, the MHC class I biding epitope is an HLA-A2-restricted epitope or an HLA-A24-restricted epitope.

In another aspect, the invention provides a method of inducing a T-cell response to a tumor that expresses PSCA, said method comprising administering to a mammal who has said tumor or who has had said tumor removed, a composition comprising a polypeptide comprising an MHC class I-binding epitope, whereby a T-cell response to PSCA is induced in the mammal, wherein the composition does not comprise a whole tumor cell. In some embodiments the tumor is a tumor that overexpresses prostate stem cell antigen relative to the normal tissue from which the tumor is derived. For example, in some embodiments, the tumor is a pancreatic cancer, a bladder cancer, or a prostate cancer. In some embodiments, the mammal is a human and the PSCA is human PSCA. In some embodiments, the MHC class I-binding epitope is an HLA-A2-restricted epitope, an HLA-A3-restricted epitope, or an HLA-A24-restricted epitope. In some embodiments, the polypeptide further comprises an MHC class II-binding epitope. In some embodiments, the polypeptide comprises a plurality of MHC class I-binding epitopes of PSCA. In some embodiments, the polypeptide comprises a plurality of MHC class I-binding epitopes which bind allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the T-cell response comprises induction of PSCA specific CD8+ T cells. In some embodiments, the T-cell response further comprises induction of PSCA specific CD4+ cells. In some embodiments, the composition further comprises an adjuvant or a non-PSCA antigen. In some embodiments, the composition is administered in an amount sufficient to induce tumor regression or inhibit progression of a cancer in the mammal. In some embodiments, the composition is administered in an amount sufficient to delay or prevent recurrence of cancer in the mammal, wherein the mammal has had the tumor removed. In some embodiments, the composition is acellular. In some embodiments, the composition comprises a recombinant vector comprising a bacterium (e.g., Listeria monocytogenes), virus or yeast expressing the polypeptide.

In another aspect, the invention provides a method of inducing a T-cell response to a tumor that expresses PSCA, said method comprising administering to a mammal who has said tumor or who has had said tumor removed, a composition comprising a polynucleotide encoding a polypeptide comprising an MHC class I-binding epitope, whereby a T-cell response to PSCA is induced in the mammal, wherein the composition does not comprise a whole tumor cell. In some embodiments the tumor is a tumor that overexpresses prostate stem cell antigen relative to the normal tissue from which the tumor is derived (e.g., a pancreatic cancer, a bladder cancer or a prostate cancer). In some embodiments, the mammal is a human and the PSCA is human PSCA. In some embodiments, the MHC class I-binding epitope is an HLA-A2-restricted epitope, an HLA-A3-restricted epitope, or an HLA-A24-restricted epitope. In some embodiments, the polypeptide further comprises an MHC class II-binding epitope. In some embodiments, the polypeptide comprises a plurality of MHC class I-binding epitopes. In some embodiments, the polypeptide comprises a plurality of MHC class I-binding epitopes which bind allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the T-cell response comprises induction of PSCA specific CD8+ T cells. In some embodiments, the T-cell response further comprises induction of PSCA specific CD4+ cells. In some embodiments, the composition further comprises an adjuvant or a non-PSCA antigen. In some embodiments, the composition is administered in an amount sufficient to induce tumor regression or inhibit progression of a cancer in the mammal. In some embodiments, the composition is administered in an amount sufficient to delay or prevent recurrence of cancer in the mammal, wherein the mammal has had the tumor removed. In some embodiments, the composition is acellular. In some embodiments, the composition comprises a recombinant vector comprising a bacterium (e.g., Listeria monocytogenes), virus or yeast comprising the polynucleotide and expressing the polypeptide.

In still another aspect, the invention provides a method of treating cancer in a mammal who has a PSCA-expressing tumor or who has had a PSCA-expressing tumor removed, comprising: administering to the mammal a composition comprising a polypeptide comprising an MHC class I-binding epitope, whereby a T-cell response to PSCA is induced in the mammal, wherein the composition does not comprise a whole tumor cell; and further treating the mammal with chemotherapy, radiation, surgery, hormone therapy, or additional immunotherapy.

In another aspect, the invention provides a method of treating cancer in a mammal who has a PSCA-expressing tumor or who has had a PSCA-expressing tumor removed, comprising: administering to the mammal a composition comprising a polynucleotide encoding a polypeptide comprising an MHC class I-binding epitope, whereby a T-cell response to PSCA is induced in the mammal, wherein the composition does not comprise a whole tumor cell; and further treating the mammal with chemotherapy, radiation, surgery, hormone therapy, or additional immunotherapy.

In still another aspect, the invention provides a method of generating a T-cell response in a mammal to a tumor that expresses prostate stem cell antigen (PSCA), said method comprising administering to a mammal who has said tumor or who has had said tumor removed, an effective amount of a composition comprising a PSCA-specific CD8+ T cell population.

In a further aspect, the invention provides a method of identifying a composition as being useful in an antitumor vaccine, comprising testing lymphocytes of a mammal to whom the composition has been administered to determine if said lymphocytes comprise PSCA specific CD8+ T cells, wherein the presence of PSCA specific CD8+ T-cells indicates that the composition is useful in an antitumor vaccine.

In another aspect, the invention provides a method of assessing if a mammal is having a favorable response to an antitumor vaccine, comprising testing lymphocytes of a mammal to whom the composition has been administered to determine if said lymphocytes comprise PSCA specific CD8+ T cells, wherein the presence of PSCA specific CD8+ T-cells indicates that the mammal is having a favorable response to the antitumor vaccine.

In another aspect, the invention provides a vaccine that induces a CD8+ T cell response to PSCA-expressing tumor cell in a human, comprising: a polypeptide comprising an MHC class I-binding epitope of human PSCA, wherein the vaccine is not a whole tumor cell. In some embodiments, the vaccine further comprises an adjuvant.

In a still further aspect, the invention provides a vaccine that induces a CD8+ T cell response to a PSCA-expressing tumor cell in a human, comprising a polynucleotide encoding a polypeptide comprising an MHC class I-binding epitope of human PSCA, wherein the vaccine is not a whole tumor cell. In some embodiments, the vaccine further comprises an adjuvant.

In another aspect, the invention provides an immunogenic composition that induces a T cell response to a PSCA-expressing tumor cell in a mammal, comprising a polypeptide comprising an MHC class I-binding epitope of PSCA. In some embodiments, the immunogenic composition does not comprise a whole mammalian cell. In some embodiments, the immunogenic composition comprises a recombinant vector comprising a bacterium, virus or yeast expressing the polypeptide. In some embodiments, the polypeptide further comprises an MHC class II-binding epitope. In some embodiments, the polypeptide comprises a plurality of MHC class I-binding epitopes of PSCA. In some embodiments, the polypeptide comprises a plurality of MHC class I-binding epitopes of PSCA that bind the allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the T-cell response comprises induction of PSCA specific CD8+ T cells. In some embodiments, the immunogenic composition comprises an adjuvant or a non-PSCA antigen. In some embodiments, the polypeptide does not comprise PSCA, but rather comprises a fragment of PSCA, wherein the fragment comprises the MHC class I epitope. In some embodiments, the fragment is at least 8 amino acids in length.

In a further aspect, the invention provides an immunogenic composition that induces a T cell response to a PSCA-expressing tumor cell in a mammal, comprising: a polynucleotide encoding a polypeptide comprising an MHC class I-binding epitope of PSCA. In some embodiments, the immunogenic composition does not comprise a whole mammalian cell. In some embodiments, the immunogenic composition comprises a bacterium, virus or yeast comprising the polynucleotide and expressing the polypeptide. In some embodiments, the polypeptide further comprises an MHC class II-binding epitope of PSCA. In some embodiments, the polypeptide comprises a plurality of MHC class I-binding epitopes of PSCA. In some embodiments, the polypeptide comprises a plurality of MHC class I-binding epitopes of PSCA that bind the allelic forms of MHC class I that are expressed by the mammal. In some embodiments, the polypeptide comprises PSCA. In some embodiments, the polypeptide comprises a fragment of PSCA. In some embodiments, the polypeptide consists of PSCA, or a fragment thereof. In some embodiments, the immunogenic composition further comprises an adjuvant or a non-PSCA antigen. In some embodiments, the polypeptide comprises a fragment of PSCA, wherein the fragment comprises the MHC class I-binding epitope and is at least 8 amino acids in length.

In another aspect, the invention provides an immunogenic composition that induces a T cell response to a PSCA-expressing tumor cell in a mammal, comprising a polypeptide comprising an MHC class II-binding epitope of PSCA, wherein the immunogenic composition does not comprise a whole mammalian cell.

In an additional aspect, the invention provides an immunogenic composition that induces a T cell response to a PSCA-expressing tumor cell in a mammal, comprising: a polynucleotide encoding a polypeptide comprising an MHC class II-binding epitope of PSCA, wherein the immunogenic composition does not comprise a whole mammalian cell.

In still another aspect, the invention provides a method of inducing a T-cell response in a mammal to a tumor that expresses prostate stem cell antigen (PSCA), said method comprising administering to a mammal who has said tumor or who has had said tumor removed, a composition comprising a polypeptide comprising a PSCA variant, wherein the PSCA variant has at least about an 70% sequence identity to PSCA, or to a fragment of PSCA that comprises an MHC class I-binding epitope of PSCA, whereby a CD8+ T-cell response to PSCA is induced in the mammal. In some embodiments, the composition does not comprise a whole tumor cell. In some embodiments, the fragment of PSCA is at least about 9 amino acids in length. In some embodiments, the tumor overexpresses prostate stem cell antigen relative to a normal tissue from which the tumor is derived. In some embodiments, the tumor is a pancreatic cancer, a bladder cancer or a prostate cancer. In some embodiments, the mammal is a human and the PSCA is human PSCA. In some embodiments, the PSCA is SEQ ID NO:22.

In another aspect, the invention provides a method of inducing a T-cell response to a tumor that expresses prostate stem cell antigen (PSCA) in a mammal, said method comprising administering to the mammal who has said tumor or who has had said tumor removed, a composition comprising a polynucleotide encoding a polypeptide comprising a PSCA variant, wherein the PSCA variant has at least about a 70% sequence identity to PSCA or to a fragment of PSCA that comprises an MHC class I-binding epitope of PSCA, whereby a CD8+ T-cell response to PSCA is induced in the mammal. In some embodiments, the composition does not comprise a whole tumor cell. In some embodiments, the fragment of PSCA is at least about 9 amino acids in length. In some embodiments, the tumor overexpresses prostate stem cell antigen relative to a normal tissue from which the tumor is derived. In some embodiments, the tumor is a pancreatic cancer, a bladder cancer or a prostate cancer. In some embodiments, the mammal is a human and the PSCA is human PSCA. In some embodiments, the PSCA is SEQ ID NO:22.

In still another aspect, the invention provides an immunogenic composition that induces a CD8+ T cell response to a PSCA-expressing tumor cell in a mammal, comprising a polypeptide comprising a PSCA variant, wherein the PSCA variant has at least about an 70% sequence identity to PSCA, or to a fragment of PSCA that comprises an MHC class I-binding epitope of PSCA. In some embodiments, composition does not comprise a whole tumor cell. In some embodiments, the mammal is a human and the PSCA is human PSCA. In some embodiments, the PSCA is SEQ ID NO:22.

In yet another aspect, the invention provides an immunogenic composition that induces a CD8+ T cell response to a PSCA-expressing tumor cell in a mammal, comprising a polynucleotide encoding a polypeptide comprising a PSCA variant, wherein the PSCA variant has at least about a 70% sequence identity to PSCA or to a fragment of PSCA that comprises an MHC class I-binding epitope of PSCA. In some embodiments, the composition does not comprise a whole tumor cell. In some embodiments, the mammal is a human and the PSCA is human PSCA. In some embodiments, the PSCA is SEQ ID NO:22.

In some embodiments of each of the aforementioned aspects, as well as other aspects described herein, the MHC class I-binding epitope binds to an allelic form of MHC class I which is expressed by the mammal to which it is administered. Likewise, in some embodiments of each of the aforementioned aspects, as well as other aspects described herein, the MHC class II-binding epitope binds to an allelic form of MHC class II which is expressed by the mammal to which it is administered.

In some embodiments of each of the aforementioned aspects, as well as other aspects described herein, wherein the polypeptide comprises an MHC class I-binding epitope, the T-cell response is a CD8+ T-cell response. In some other embodiments wherein the polypeptide comprises an MHC class II-binding epitope, the T-cell response is a CD4+ T-cell response.

In some embodiments of each of the aforementioned aspects as well as the other aspects described herein, the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In some embodiments of each of the aforementioned aspects as well as the other aspects described herein, the polypeptide comprising the MHC class I-binding epitope comprises at least two, at least three, at least four, at least five, or at least six epitopes selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments of each of the aspects, the polypeptide comprises each of the aforementioned seven epitopes.

In some embodiments of each of the aforementioned aspects as well as the other aspects described herein, the polypeptide comprising the MHC class I-binding epitope comprises a fusion protein. In some embodiments, the fusion protein comprises a first and a second portion, wherein the first portion comprises an MHC class I-binding epitope of PSCA, and wherein the second portion comprises a sequence of at least 6 amino acid residues, wherein the sequence of said second portion is not in PSCA.

In some embodiments of each of the aforementioned aspects, as well as other aspects described herein, the polypeptide comprising the MHC class I-binding epitope and/or MHC class II-binding epitope comprises PSCA (e.g., human PSCA), or a fragment thereof.

In an additional aspect, the invention provides an isolated antibody, or fragment thereof, that binds to an epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In addition, in another aspect, the invention further provides a method of inducing a T-cell response to a tumor that expresses prostate stem cell antigen (PSCA), said method comprising administering to a mammal who has said tumor or who has had said tumor removed, a composition comprising a polypeptide and/or a polynucleotide encoding a polypeptide, wherein the polypeptide comprise a fragment of PSCA comprising an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), whereby a T-cell response to PSCA is induced in the mammal. In some embodiments, the composition does not comprise a whole tumor cell. In some embodiments, the tumor overexpresses prostate stem cell antigen relative to a normal tissue from which the tumor is derived. In some embodiments, the mammal is a human. In some embodiments, the fragment of PSCA is at least 10 amino acids in length, or at least 26 amino acids in length. In some embodiments, the polypeptide comprises a fusion protein, wherein the fusion protein comprises a first and a second portion, wherein the first portion comprises the fragment of PSCA, and wherein the second portion comprises a sequence of at least 6 amino acid residues, wherein the sequence of said second portion is not in PSCA. In some embodiments, the T-cell response comprises induction of PSCA-specific CD8+ T cells. In some embodiments, the composition is administered in an amount sufficient to induce tumor regression or an amount sufficient to inhibit progression of a cancer in the mammal. In some embodiments, the composition is administered in an amount sufficient to delay or prevent recurrence of cancer in the mammal, wherein the mammal has had said tumor removed. In some embodiments, the composition comprises a recombinant vector comprising a bacterium, virus or yeast expressing the polypeptide. In some embodiments, the vaccine comprises a whole cell from a tumor cell line that has been selected or modified to overexpress the polypeptide relative to the tumor cell line prior to selection or modification.

In another aspect, the invention provides a method of treating cancer in a mammal who has a PSCA-expressing tumor or who has had a PSCA-expressing tumor removed, comprising: administering to the mammal a composition comprising a polypeptide and/or a polynucleotide encoding a polypeptide, wherein the polypeptide comprises a fragment of PSCA that comprises an MHC class I-binding epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), whereby a T-cell response to PSCA is induced in the mammal. In some embodiments, the composition does not comprise a whole tumor cell. In some embodiments, the method further comprises treating the mammal with chemotherapy, radiation, surgery, hormone therapy, or additional immunotherapy. In some embodiments, the vaccine comprises a whole cell from a tumor cell line that has been selected or modified to overexpress the polypeptide relative to the tumor cell line prior to selection or modification.

In still another aspect, the invention provides an immunogenic composition that induces a T cell response to a PSCA-expressing tumor cell in a mammal, comprising: a polypeptide and/or a polynucleotide encoding a polypeptide, wherein the polypeptide comprises a fragment of PSCA that comprises an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the immunogenic composition does not comprise a whole mammalian cell. In some embodiments, the composition does not comprise a whole tumor cell. In some embodiments, the tumor overexpresses prostate stem cell antigen relative to a normal tissue from which the tumor is derived. In some embodiments, the mammal is a human. In some embodiments, the fragment of PSCA is at least 10 amino acids in length, or at least 26 amino acids in length. In some embodiments, the polypeptide comprises a fusion protein, wherein the fusion protein comprises a first and a second portion, wherein the first portion comprises the fragment of PSCA, and wherein the second portion comprises a sequence of at least 6 amino acid residues, wherein the sequence of said second portion is not in PSCA. In some embodiments, the T-cell response comprises induction of PSCA-specific CD8+ T cells. In some embodiments, the composition comprises a recombinant vector comprising a bacterium, virus or yeast expressing the polypeptide. In some embodiments, the vaccine comprises a whole cell from a tumor cell line that has been selected or modified to overexpress the polypeptide relative to the tumor cell line prior to selection or modification.

In an additional aspect, the invention provides a vaccine that induces a T cell response to a PSCA-expressing tumor cell in a human, comprising: a polypeptide and/or a polynucleotide encoding a polypeptide, wherein the polypeptide comprises a fragment of PSCA comprising an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9); and an adjuvant. In some embodiments, the vaccine does not comprise a whole tumor cell. In some embodiments, the vaccine comprises a whole cell from a tumor cell line that has been selected or modified to overexpress a polypeptide relative to the tumor cell line prior to selection or modification.

In further aspects, the invention provides polypeptides, polynucleotides, expression vectors for use in the compositions and methods described herein. The invention also further provides methods of making the polypeptides, polynucleotides, expression vectors, compositions, and vaccines described herein.

In one aspect, the invention provides a polypeptide comprising a fragment of PSCA, wherein the fragment of PSCA is at least 8 amino acids in length and comprises an MHC class I epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the fragment is at least 10 amino acids in length or at least 26 amino acids in length. Polynucleotides encoding the polypeptide are also provides, as are cells comprising the polypeptide and/or polynucleotide. The invention further provides a recombinant vector comprising a bacterium, virus, or yeast comprising the polynucleotide, and expressing the polypeptide.

In another aspect, the invention provides a polypeptide comprising a fusion protein that comprises a first and a second portion, wherein the first portion comprises PSCA, or fragment thereof that comprises an MHC class I epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), and wherein the second portion comprises a sequence of at least 6 amino acid residues, wherein the sequence of said second portion is not in PSCA. In some embodiments, the first portion comprises PSCA. In some alternative embodiments, the first portion comprises a fragment of PSCA. In some embodiments, the fragment is at least 10 amino acids in length or at least 26 amino acids in length. Polynucleotides encoding the polypeptide are also provides, as are cells comprising the polypeptide and/or polynucleotide. The invention further provides a recombinant vector comprising a bacterium, virus, or yeast comprising the polynucleotide, and expressing the polypeptide. Immunogenic compositions and vaccines comprising the polypeptide and/or polynucleotide are further provided. Methods of inducing a T-cell response to a tumor that expresses PSCA and methods of treating cancer in a mammal who has a PSCA-expressing tumor or who has had a PSCA-expressing tumor removed, comprising administering to the mammal a composition comprising the polypeptide and/or polynucleotide, whereby a T-cell response to PSCA is induced in the mammal. In some embodiments, the T-cell response is a PSCA specific CD8+ T-cell response.

The invention further provides the use of each of the compositions described herein (e.g., in any of the aspects above, or in the Detailed Description of the invention or Examples, below) in a pharmaceutical composition or in the manufacture of a medicament. The pharmaceutical composition/medicament may be used in any of the methods described herein. For example, the invention provides the use of each of the compositions described herein for the manufacture of a pharmaceutical composition for such uses as inducing a T-cell response to a tumor that expresses PSCA. The invention also provides the use of each of the compositions described herein for the manufacture of a pharmaceutical composition for treating cancer in a mammal (e.g., a mammal who has a PSCA-expressing tumor or who has had a PSCA-expressing tumor removed).

### BRIEF DESCRIPTIONS OF THE DRAWINGS

FIG. 1 shows a T2 binding assay identifying PSCA protein derived epitopes that bind to HLA-A2, A3, and A24 molecules. T2 cells were pulsed with 225 micrograms of peptide per ml overnight at room temperature before analysis by flow cytometry. T2 cells expressing HLA-A2 (A) or HLA-A24 (C) were stained with an unlabeled mouse anti-HLA class I molecule monoclonal antibody W6/32 and a goat-anti-mouse FITC-labeled IgG2a secondary antibody. T2 cells genetically modified to express A3 (B) were stained with an unlabeled mouse anti-human HLA-A3 specific monoclonal antibody GAPA3 and a FITC-labeled IgG2a secondary antibody. The Mesothelin A1(309-317) (EIDESLIFY) (SEQ ID NO:1) peptide was used as a non-binding negative control.

FIG. 2 shows expression of surface PSCA on Panc 6.03 and Panc 10.05 vaccine lines. The pancreatic tumor vaccine lines Panc 6.03 and Panc 10.05 were analyzed by flow cytometry for their levels of surface PSCA using the PSCA specific monoclonal antibody 1G8 as the primary antibody and goat anti-mouse IgG FITC as the secondary antibody. The solid line represents the isotype control and the shaded area represents PSCA staining.

FIG. 3A to 3D shows an ELISPOT analysis of CD8+ T cells from PBMCs before and shortly after vaccination. Initially, no post-vaccination induction was observed of PSCA-specific T cells in DTH responders or non-DTH responders who received an allogeneic GM-CSF-secreting tumor vaccine for pancreatic cancer. FIG. 3A. ELISPOT analysis of PBL from two patients who were HLA-A 2 and HLA-A3 positive (DTH Responder Patient 2.38 (top four in figure legend) and DTH Non-Responder Patient 2.18 (bottom four in figure legend)); FIG. 3B. ELISPOT analysis of PBL from two patients who were HLA-A3 positive (DTH Responder Patient 2.71 (top seven in figure legend) and DTH Non-Responder Patient 2.62 (bottom seven in figure legend)); FIG. 3C. ELISPOT analysis of PBL from two-patients who were HLA-A24 positive (DTH Responder Patient 2.73 (top six in figure legend) and DTH Non-Responder Patient 2.22 (bottom six in figure legend)). FIG. 3D. ELISPOT analysis of PBL from eight patients who were non-responders. ELISPOT analysis for IFN-.gamma.-expressing cells was performed using PBMC that were isolated on the day prior to vaccination or 28 days following each of the vaccinations. Lymphocytes were isolated by ficoll-hypaque separation and stored frozen in liquid nitrogen until the day of assay. CD8+ T cell enrichment was performed prior to analysis. T2-A3 cells were pulsed with the six PSCA derived epitopes as indicated. Negative HIV-NEFA3 (94-103) values were subtracted out. T2-A2 cells were pulsed with the three PSCA derived epitopes as indicated. Negative HIV-GAG(77-85) values were subtracted out. T2-A24 cells were pulsed with the five PSCA derived epitopes as indicated. Negative Tyrosinase A24(206-214) values were subtracted. For the detection of nonspecific background, the number of IFN-.gamma. spots for CD8+ T cells specific for the irrelevant control peptides were counted. The HLA-A2 binding HIV-GAG protein derived epitope (SLYNTVATL) (SEQ ID NO:2), the HLA-A3 binding HIV-NEF protein derived epitope (QVPLRPMTYK) (SEQ ID NO:3), and the HLA-A24 binding tyrosinase protein derived epitope (AFLPWHRLF) (SEQ ID NO:4) were used as negative control peptides in these assays. Data represents the average of each condition assayed in triplicate and standard deviations were less than 5%. The number of human interferon gamma (hIFNg) spots per 10⁵ CD8+ T cells is plotted. Analysis of each patient's PBL was performed at least twice.

FIG. 4 shows an ELISPOT analysis of CD8+ T cells from PBMCs of DTH Responder Patient 2.38 four years post completion of treatment. No induction was observed of PSCA-specific T cells.

FIG. 5 shows an ELISPOT analysis of CD8+ T cells from PBMCs of DTH Responder Patient 2.71 four years post completion of treatment. Significant induction of PSCA-specific T cells was observed.

FIG. 6 shows an ELISPOT analysis of CD8+ T cells from PBMCs of DTH Responder Patient 2.73 four years post completion of treatment. Significant induction of PSCA-specific T cells was observed.

FIG. 7 shows an ELISPOT analysis of CD8+ T cells from PBMCs of DTH Responder Patient 2.38 four years post completion of treatment. No induction was observed of PSCA-specific T cells. (This was a repetition of the results shown in FIG. 4.)

FIG. 8 shows an ELISPOT analysis of CD8+ T cells from PBMCs of DTH Responder Patient 2.71 four years post completion of treatment. Significant induction of PSCA-specific T cells was observed. (This was a repetition of the results shown in FIG. 5.)

FIG. 9 shows an ELISPOT analysis of CD8+ T cells from PBMCs of DTH Responder Patient 2.73 four years post completion of treatment. Significant induction of PSCA-specific T cells was observed. (This was a repetition of the results shown in FIG. 6.)

FIG. 10 shows the nucleotide sequence of human PSCA (SEQ ID NO:20) that has been derived from analysis of genomic human DNA (GenBank Acc. No. BC048808). The start codon is BOLD and underlined.

FIG. 11 shows the nucleotide sequence (GenBank Acc. No. BC065183) (SEQ ID NO:21) of human PSCA derived from analysis of cDNA (not from human genomic DNA). The start codon is BOLD and underlined.

FIG. 12 shows the protein sequence of human PSCA (SEQ ID NO:22). The underlined amino acids at the beginning of the sequence represent the amino-terminal hydrophobic signal sequence. The underlined amino acids at the end of the sequence represent the C-terminal GPI-anchoring sequences.

### DETAILED DESCRIPTION OF THE INVENTION

The present invention relates, in some aspects, to the identification of prostate stem cell antigen (PSCA) as an immunologically relevant tumor antigen. PSCA has been identified as a tumor antigen against which T cell responses are elicited following vaccination with a GM-CSF gene modified allogeneic pancreatic cancer vaccine. Specifically, T cell responses against peptides derived from an antigen, prostate stem cell antigen (PSCA), which is demonstrated by gene expression analysis to be overexpressed in pancreatic cancer relative to normal pancreatic tissue and other normal tissues (Argani et al., Cancer Research, 61:4320-4324 (2001)), was accessed in pancreatic cancer patients which were treated with an allogeneic pancreatic tumor cell line engineered to express GM-CSF. (See Jaffee et al., J. of Clinical Oncology, 19:145-156 (2001) and US Publication No. 2005/0175625, each of which is hereby incorporated by reference herein in its entirety.) HLA binding peptides corresponding to HLA alleles expressed by the treated patients (A2, A3 and A24) were synthesized and utilized in a quantitative Elispot assay. It was found that multiple HLA A2 binding peptides as well two HLA A3 and two HLA 24 binding peptides from PSCA were, in fact, recognized by T cells from vaccinated pancreatic cancer patients expressing the appropriately matched HLA alleles. Specifically, in 2 of 3 patients demonstrating a clinical response to the pancreatic cancer vaccine, there was an increase in T cell precursor frequency to the appropriate HLA PSCA peptide of greater than five-fold post vaccination. In contrast, patients receiving comparable doses of vaccine but who did not demonstrate clinical responses failed to demonstrate a significant increase in frequency of T cells responding to PSCA post vaccine. Therefore, there was a good correlation between clinical response to the genetically modified whole cell vaccine and a vaccine induced increase in T cell responses to PSCA as measured with the quantitative Elispot assay. These results define PSCA as a relevant target for the generation of anti-tumor immune responses as well as a relevant marker for the generation of anti-tumor immune responses.

In some embodiments, the PSCA is incorporated into immunotherapy through formulation of multiple types of vaccines including peptide-based vaccines and recombinant vaccines in which the PSCA gene is incorporated into nucleic acid based vaccines, recombinant viral vaccines (such as vaccinia virus, cow pox, canary pox, adenovirus, modified vaccinia ancra, Venezuelan equine encephalitis virus etc.), recombinant bacterial vaccines (such as recombinant Listeria, recombinant Salmonela, recombinant Shigella) and recombinant yeast vaccines. In some embodiments, immune responses to PSCA are generated by introduction of the PSCA gene into the hematopoietic stem cells followed by transplantation and administration of systemic dendritic cell activators. In some embodiments, the PSCA antigen, as protein, gene, or specific HLA restricted peptides, could be used to generate PSCA specific T cell lines and clones from patients in vitro which are then adoptively transferred into patients with cancer. In another embodiment, PSCA specific monoclonal antibodies are utilized to treat patients with cancers overexpressing PSCA. Alternatively, in some embodiments, T cell receptors cloned from PSCA specific T cells can be introduced into vectors and then subsequently introduced into autologus T cells generating PSCA specific T cell populations. In some embodiments, PSCA peptides, protein or gene could be used to load antigen presenting cells (specifically dendritic cells) which are utilized to immunize patients with cancer.

Alternatively, in some embodiments, PSCA is utilized as a marker for testing various cancer vaccines and other immunotherapies. This can be done by utilizing either the gene and appropriate vector, protein or peptides to load antigen presenting cells which would be utilized to stimulate T cells in intracellular cytokine assays, chromium release assays or quantitative Elispot assays. In addition, in some embodiments, identified PSCA peptides can be used to load the restricting HLA molecules in the form of dimers or tetramers which could be utilized as reagents to monitor the frequency and cell surface markers and functional status of PSCA specific T cells using flow cytometric staining.

In one aspect, the invention provides a method of inducing a T-cell response to a tumor that expresses prostate stem cell antigen (PSCA), said method comprising administering to a mammal who has said tumor or who has had said tumor removed, a composition comprising a polypeptide comprising an MHC class I-binding epitope and/or an MHC class II-binding, epitope, whereby a T-cell response to PSCA is induced in the mammal. In some embodiments, the composition does not comprise a whole tumor cell.

In another aspect, the invention provides a method of inducing a T-cell response to a tumor that expresses prostate stem cell antigen (PSCA), said method comprising administering to a mammal who has said tumor or who has had said tumor removed, a composition comprising a polynucleotide encoding a polypeptide comprising an MHC class I-binding epitope and/or an MHC class II-binding epitope, whereby a T-cell response to PSCA is induced in the mammal. In some embodiments, the composition does not comprise a whole tumor cell.

In another aspect, the invention provides a method of treating cancer in a mammal comprising administering to the mammal a composition comprising a polypeptide comprising an MHC class I-binding epitope and/or an MHC class II-binding epitope, whereby a T-cell response to PSCA is induced in the mammal.

In another aspect, the invention provides a method of treating cancer in a mammal who has a PSCA-expressing tumor or who has had a PSCA-expressing tumor removed, comprising: administering to the mammal a composition comprising a polypeptide comprising an MHC class I-binding epitope and/or an MHC class II-binding epitope, whereby a T-cell response to PSCA is induced in the mammal; and further treating the mammal with chemotherapy, radiation, surgery, hormone therapy, or additional immunotherapy. In some embodiments, the composition does not comprise a whole tumor cell.

In still another aspect, the invention provides a method of treating cancer in a mammal comprising administering to the mammal a composition comprising a polynucleotide encoding a polypeptide comprising an MHC class I-binding epitope and/or an MHC class II-binding epitope, whereby a T-cell response to PSCA is induced in the mammal.

In another aspect, the invention provides a method of treating cancer in a mammal who has a PSCA-expressing tumor or who has had a PSCA-expressing tumor removed, comprising: administering to the mammal a composition comprising a polynucleotide encoding a polypeptide comprising an MHC class I-binding epitope and/or an MHC class II-binding epitope, whereby a T-cell response to PSCA is induced in the mammal; and further treating the mammal with chemotherapy, radiation, surgery, hormone therapy, or additional immunotherapy. In some embodiments, the composition does not comprise a whole tumor cell.

In still another aspect, the invention provides a vaccine that induces a T cell response to a PSCA-expressing tumor cell in a human, comprising: a polypeptide comprising an MHC class I-binding epitope and/or an MHC class II-binding epitope; and an adjuvant. In some embodiments, the vaccine does not comprise a whole tumor cell.

In still another aspect, the invention provides a vaccine that induces a T cell response to a PSCA-expressing tumor cell in a human, comprising: a polynucleotide encoding a polypeptide comprising an MHC class I-binding epitope and/or an MHC class II-binding epitope; and an adjuvant. In some embodiments, the vaccine does not comprise a whole tumor cell.

In a further aspect, the invention provides a vaccine that induces a T cell response to PSCA-expressing tumor cell in a human, comprising: a whole cell from a tumor cell line that has been selected or modified to overexpress a polypeptide relative to the tumor cell line prior to selection or modification, wherein the polypeptide comprises an MHC class I-binding epitope and/or an MHC class II-binding epitope; and an adjuvant.

In another aspect, the invention provides a method of inducing a T-cell response to a tumor that expresses prostate stem cell antigen (PSCA), said method comprising administering to a mammal who has said tumor or who has had said tumor removed, a composition comprising a whole cell from a tumor cell line that has been selected or modified to overexpress a polypeptide relative to the tumor cell line prior to selection or modification, wherein the polypeptide comprises an MHC class I-binding epitope and/or an MHC class II-binding epitope, whereby a T-cell response to PSCA is induced in the mammal.

In another aspect, the invention provides a method of inducing a T-cell response to a tumor that expresses prostate stem cell antigen (PSCA), said method comprising administering to a mammal who has said tumor or who has had said tumor removed, an effective amount of a composition comprising a polypeptide comprising an MHC class I-binding epitope, whereby a T-cell response to PSCA is induced in the mammal, wherein the composition does not comprise a whole tumor cell.

In another aspect, the invention provides a method of inducing a T-cell response to a tumor that expresses prostate stem cell antigen (PSCA), said method comprising administering to a mammal who has said tumor or who has had said tumor removed, an effective amount of a composition comprising a polynucleotide encoding a polypeptide comprising an MHC class I-binding epitope, whereby a T-cell response to PSCA is induced in the mammal, wherein the composition does not comprise a whole tumor cell.

In still another aspect, the invention provides a method of treating cancer in a mammal who has a PSCA-expressing tumor or who has had a PSCA-expressing tumor removed, comprising: administering to the mammal a composition comprising a polypeptide comprising an MHC class I-binding epitope, whereby a T-cell response to PSCA is induced in the mammal, wherein the composition does not comprise a whole tumor cell; and further treating the mammal with chemotherapy, radiation, surgery, hormone therapy, or additional immunotherapy.

In another aspect, the invention provides a method of treating cancer in a mammal who has a PSCA-expressing tumor or who has had a PSCA-expressing tumor removed, comprising: administering to the mammal a composition comprising a polynucleotide encoding a polypeptide comprising an MHC class I-binding epitope, whereby a T-cell response to PSCA is induced in the mammal, wherein the composition does not comprise a whole tumor cell; and further treating the mammal with chemotherapy, radiation, surgery, hormone therapy, or additional immunotherapy.

In still another aspect, the invention provides a method of generating a T-cell response in a mammal to a tumor that expresses prostate stem cell antigen (PSCA), said method comprising administering to a mammal who has said tumor or who has had said tumor removed, an effective amount of a composition comprising a PSCA-specific CD8+ T cell population.

In a further aspect, the invention provides a method of identifying a composition as being useful in an antitumor vaccine, comprising testing lymphocytes of a mammal to whom the composition has been administered to determine if said lymphocytes comprise PSCA specific CD8+ T cells, wherein the presence of PSCA specific CD8+ T-cells indicates that the composition is useful in an antitumor vaccine.

In another aspect, the invention provides a method of assessing if a mammal is having a favorable response to an antitumor vaccine, comprising testing lymphocytes of a mammal to whom the composition has been administered to determine if said lymphocytes comprise PSCA specific CD8+ T cells, wherein the presence of PSCA specific CD8+ T-cells indicates that the mammal is having a favorable response to the antitumor vaccine.

In another aspect, the invention provides a vaccine that induces a CD8+ T cell response to PSCA, comprising (a) a polypeptide comprising an MHC class I-binding epitope, and (b) an adjuvant or an additional tumor antigen.

In a still further aspect, the invention provides a vaccine that induces a CD8+ T cell response to PSCA, comprising a polynucleotide encoding a polypeptide comprising (a) an MHC class I-binding epitope, and (b) an adjuvant or an additional tumor antigen.

In another aspect, the invention provides an immunogenic composition that induces a T cell response to a PSCA-expressing tumor cell in a mammal, comprising a polypeptide comprising an MHC class I-binding epitope of PSCA, wherein the immunogenic composition does not comprise a whole mammalian cell.

In a further aspect, the invention provides an immunogenic composition that induces a T cell response to a PSCA-expressing tumor cell in a mammal, comprising: a polynucleotide encoding a polypeptide comprising an MHC class I-binding epitope of PSCA, wherein the immunogenic composition does not comprise a whole mammalian cell.

In another aspect, the invention provides an immunogenic composition that induces a T cell response to a PSCA-expressing tumor cell in a mammal, comprising a polypeptide comprising an MHC class II-binding epitope of PSCA, wherein the immunogenic composition does not comprise a whole mammalian cell.

In an additional aspect, the invention provides an immunogenic composition that induces a T cell response to a PSCA-expressing tumor cell in a mammal, comprising: a polynucleotide encoding a polypeptide comprising an MHC class II-binding epitope of PSCA, wherein the immunogenic composition does not comprise a whole mammalian cell.

In still another aspect, the invention provides a method of inducing a T-cell response in a mammal to a tumor that expresses prostate stem cell antigen (PSCA), said method comprising administering to a mammal who has said tumor or who has had said tumor removed, a composition comprising a polypeptide comprising a PSCA variant, wherein the PSCA variant has at least about an 70% sequence identity to PSCA, or to a fragment of PSCA that comprises an MHC class I-binding epitope of PSCA, whereby a CD8+ T-cell response to PSCA is induced in the mammal.

In another aspect, the invention provides a method of inducing a T-cell response to a tumor that expresses prostate stem cell antigen (PSCA) in a mammal, said method comprising administering to the mammal who has said tumor or who has had said tumor removed, a composition comprising a polynucleotide encoding a polypeptide comprising a PSCA variant, wherein the PSCA variant has at least about a 70% sequence identity to PSCA or to a fragment of PSCA that comprises an MHC class I-binding epitope of PSCA, whereby a CD8+ T-cell response to PSCA is induced in the mammal.

In still another aspect, the invention provides an immunogenic composition that induces a CD8+ T cell response to a PSCA-expressing tumor cell in a mammal, comprising a polypeptide comprising a PSCA variant, wherein the PSCA variant has at least about an 70% sequence identity to PSCA, or to a fragment of PSCA that comprises an MHC class I-binding epitope of PSCA.

In yet another aspect, the invention provides an immunogenic composition that induces a CD8+ T cell response to a PSCA-expressing tumor cell in a mammal, comprising a polynucleotide encoding a polypeptide comprising a PSCA variant, wherein the PSCA variant has at least about a 70% sequence identity to PSCA or to a fragment of PSCA that comprises an MHC class I-binding epitope of PSCA.

In some additional aspects, the invention provides an isolated antibody, or fragment thereof, that binds to an epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). The anti-PSCA antibodies of the invention are useful as therapeutic antibodies in the treatment of cancer, as well as in diagnostic methods (see, e.g, U.S. Pat. No. 6,541,212). The anti-PSCA antibodies disclosed herein are also useful in a variety of assays, such as in Western blots to detect the level of expression of PSCA in cells. In some embodiments, the antibodies are monoclonal antibodies. In some embodiments, the antibodies are chimeric or humanized. The production of antibodies and antibody fragments such as Fabs are routine in the art.

In another aspect, the invention further provides a method of inducing a T-cell response to a tumor that expresses prostate stem cell antigen (PSCA), said method comprising administering to a mammal who has said tumor or who has had said tumor removed, a composition comprising a polypeptide and/or a polynucleotide encoding a polypeptide, wherein the polypeptide comprise a fragment of PSCA comprising an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), whereby a T-cell response to PSCA is induced in the mammal.

In another aspect, the invention provides a method of treating cancer in a mammal who has a PSCA-expressing tumor or who has had a PSCA-expressing tumor removed, comprising: administering to the mammal a composition comprising a polypeptide and/or a polynucleotide encoding a polypeptide, wherein the polypeptide comprises a fragment of PSCA that comprises an MHC class I-binding epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), whereby a T-cell response to PSCA is induced in the mammal.

In still another aspect, the invention provides an immunogenic composition that induces a T cell response to a PSCA-expressing tumor cell in a mammal, comprising: a polypeptide and/or a polynucleotide encoding a polypeptide, wherein the polypeptide comprises a fragment of PSCA that comprises an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In an additional aspect, the invention provides a vaccine that induces a T cell response to a PSCA-expressing tumor cell in a human, comprising: a polypeptide and/or a polynucleotide encoding a polypeptide, wherein the polypeptide comprises a fragment of PSCA comprising an MHC class I-binding epitope of PSCA selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9); and an adjuvant.

In further aspects, the invention provides polypeptides, polynucleotides, expression vectors for use in the compositions and methods described herein. The invention also further provides methods of making the polypeptides, polynucleotides, expression vectors, compositions, and vaccines described herein.

In one aspect, the invention provides a polypeptide comprising a fragment of PSCA, wherein the fragment of PSCA is at least 8 amino acids in length and comprises an MHC class I epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In another aspect, the invention provides a polypeptide comprising a fusion protein that comprises a first and a second portion, wherein the first portion comprises PSCA, or fragment thereof that comprises an MHC class I epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9), and wherein the second portion comprises a sequence of at least 6 amino acid residues, wherein the sequence of said second portion is not in PSCA.

PSCA is known to be expressed in a number of tumors, such as pancreatic cancer, prostate cancer and bladder cancer. In some embodiments, the cancer is prostate cancer. In some embodiments, the cancer is pancreatic cancer. Thus, the vaccines of the invention are useful for treating at least these types of tumors. Other tumors which express PSCA similarly may also be treated similarly. The prostate cancer which is treated may be either an androgen-independent prostate cancer or an androgen dependent prostate cancer. In some embodiments, the methods described herein are used to treat patients with prostate cancers that have metastasized to the bone.

In some embodiments, the tumor is a tumor that overexpresses prostate stem cell antigen relative to the normal tissue from which the tumor is derived. PSCA has been identified as being overexpressed in a number of cancers, including prostate cancer (see, e.g., Reiter et al., PNAS, 95:1735-1740 (1998); Ross et al., American Journal of Pathology, 158: 809-816; Lam et al., Clin. Cancer Res., 11:2591-2596 (2005); Zhigang et al., World Journal of Surgical Oncology, 2:13 (2004)), pancreatic cancer (see, e.g., Argani et al., Cancer Research, 61:4320-4324 (2001); McCarthy et al., Applied Immunohistochemistry and Molecular Morphology, 11:238-243 (2003)), and bladder cancer.

In some embodiments, the vaccine or other cornpositions described herein are used in the treatment of a disease or condition associated with and/or marked by expression of PSCA in a cell. In some embodiments, the vaccine or other compositions described herein are used in the treatment of a hyperproliferative disease or condition associated with and/or marked by expression of PSCA in a cell. In some embodiments, the expression is abnormal expression or overexpression of PSCA. In some embodiments, the disease or condition is a precancerous condition.

In some embodiments, the vaccine or other compositions described herein are used in the treatment of a disease or condition (e.g., a hyperproliferative disease or condition and/or a disease or condition associated with and/or marked by expression of PSCA) that is not cancer.

In some embodiments, the vaccines or other compositions of the present invention comprise a polypeptide comprising at least one MHC class I-binding epitope or at least one MHC class II-binding epitope. Alternatively, the vaccines of the present invention optionally comprise a polynucleotide encoding a polypeptide comprising at least one MHC class I-binding epitope or at least one MHC class II-binding epitope. Optionally, the polypeptides of the vaccines (or the polypeptides encoded by the polynucleotides of the vaccines) comprise a plurality of MHC class I-binding epitopes of PSCA and/or MHC class II-binding epitopes of PSCA. The multiple epitopes of the polypeptides may bind the same or different MHC allelic molecules. In one embodiment, the epitopes of the polypeptide bind a diverse variety of MHC allelic molecules.

In some embodiments, the polypeptide comprises an MHC class I-binding epitope of PSCA. In some embodiments, the polypeptide comprises a plurality of MHC class I-binding epitopes of PSCA. In some embodiments, the polypeptide comprises at least two, at least three, at least four, at least five, or at least six MHC class I-binding epitopes of PSCA.

While MHC class I-binding epitopes are effective in the practice of the present invention, MHC class II-binding epitopes can also be used. The former are useful for activating CD8+ T cells and the latter for activating CD4+ T cells. In some embodiments, the polypeptides comprise an MHC class II-binding epitope of PSCA. In some embodiments, the polypeptide comprises a plurality of MHC class II-binding epitopes.

In some embodiments, the polypeptide in the vaccine or composition or the polypeptide encoded by the polynucleotide in the vaccine or composition, comprises an MHC class I-binding epitope (or a plurality of MHC class I-binding epitopes) of PSCA, and further comprises an MHC class II-binding epitope of PSCA.

Publicly available algorithms can be used to select epitopes that bind to MHC class I and/or class II molecules. For example, the predictive algorithm “BIMAS” ranks potential HLA binding epitopes according to the predictive half-time disassociation of peptide/HLA complexes (Parker et al., J. Immunol., 152: 163-175 (1994)). The “SYFPEITHI” algorithm ranks peptides according to a score that accounts for the presence of primary and secondary HLA-binding anchor residues (Rammensee et al., Immunogenetics, 50: 213-219 (1999)). (See also, Lu et al., Cancer Research 60, 5223-5227 (2000).) Both computerized algorithms score candidate epitopes based on amino acid sequences within a given protein that have similar binding motifs to previously published HLA binding epitopes. Other algorithms can also be used to identify candidates for further biological testing.

In certain embodiments, the MHC class I- and/or MHC class II-binding epitopes are selected using an algorithm. In certain embodiments, the epitopes are selected using two algorithms.

In some embodiments, polypeptides for immunization to raise a cytolytic T cell response are optionally from 8 to 25 amino acid residues in length. Although nonamers are specifically disclosed herein, any 8 contiguous amino acids of the nonamers can be used as well. The polypeptides can be fused to other such epitopic polypeptides, or they can be fused to carriers, such as B-7, interleukin-2, or interferon-gamma. The fusion polypeptide can be made by recombinant production or by chemical linkage, e.g., using heterobifunctional linking reagents. Mixtures of polypeptides can be used. These can be mixtures of epitopes for a single allelic type of an MHC molecule, or mixtures of epitopes for a variety of allelic types. The polypeptides can also contain a repeated series of an epitope sequence or different epitope sequences in a series.

The effectiveness of an MHC class I-binding epitope or an MHC class II-binding epitope as an immunogen in a vaccine or other composition described herein can be evaluated by assessing whether a peptide comprising the epitope is capable of activating T-lymphocytes from an individual having a successful immunological response to a tumor that overexpresses PSCA (relative to normal tissue from which the tumor is derived), when the peptide is bound to an MHC molecule on an antigen-presenting cell and contacted with the T-lymphocytes under suitable conditions and for a time sufficient to permit activation of T-lymphocytes. A specific example of such an assessment is illustrated in Examples 1-2 and 4, below.

The ability of epitopes identified by one or more algorithms to bind the relevant MHC class I or MHC class II molecules can be confirmed using techniques standard in the art. Peptides corresponding to the epitopes can be prepared by synthetic methods and then tested. Alternatively, synthetic peptides or a peptide library may be screened using the assays. ELISPOT assays or by intracellular cytokine staining are among the assays that can then be used for enumeration and characterization of the antigen-specific CD8+ and/or CD4+ T cells (Lalvani et al. J. Exp. Med., 186:859-865 (1997) and Waldrop et al. J. Clin Invest., 99:1739-1750 (1997)). CD4+ T-cell proliferation assays known in the art may also be used to assess immunogenicity. Exemplary protocols for such assays are provided in, e.g., U.S. Publication No. 2005/0281783, incorporated by reference herein in its entirety.

In some embodiments, the vaccines or other compositions of the invention comprise PSCA. In some embodiments, the vaccines or other compositions of the invention comprise human PSCA. In some embodiments, they comprise polypeptides comprising at least one MHC class I-binding epitope and/or MHC class II-binding epitope (e.g., human PSCA). In some embodiments, the polypeptides are fragments of PSCA.

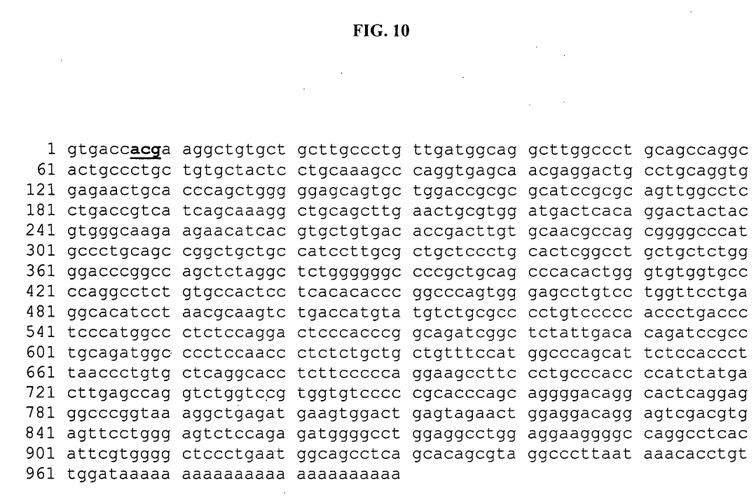

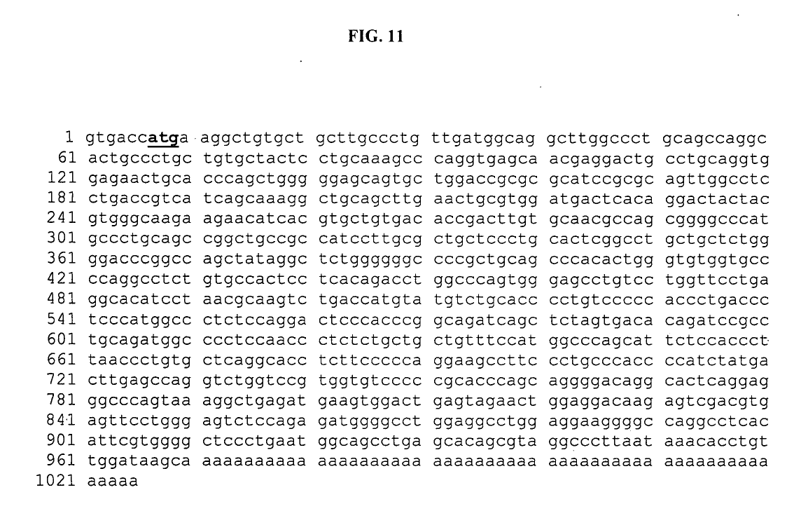

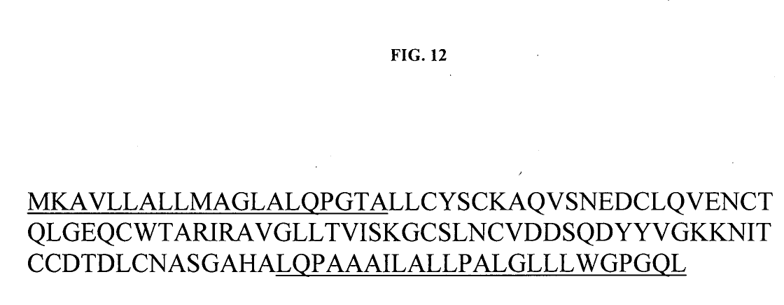

The human PSCA sequence is disclosed in, e.g., GenBank Acc. Nos. BC048808; AF043498; BC065183; and BC023582. The amino acid sequence of human PSCA is also reported in, e.g., Reiter, et al. (1998) Proc. Natl. Acad. Sci. USA 95:1735-1740). FIGS. 10 and 11 provide sequences of polynucleotides encoding human PSCA. FIG. 12 provides a sequence of human PSCA (SEQ ID NO:22). Thus, in some embodiments, the polypeptide used in the compositions and methods described herein (or encoded by the polynucleotide used in the compositions and methods described herein), comprises, or consists of, the human PSCA shown in FIG. 12 (SEQ ID NO:22). In some embodiments, the polynucleotide encoding the polypeptide comprises the coding sequence of SEQ ID NO:20 (FIG. 10) or SEQ ID NO:21 (FIG. 11).

In some embodiments, the PSCA is a murine PSCA homologue or other mammalian homologue (U.S. Pat. No. 6,979,730 and Reiter et al., PNAS, 95: 1735-1740 (1998), each of which is incorporated by reference herein in their entirety).

The vaccines (or other compositions) of the invention optionally comprise PSCA or a polynucleotide encoding PSCA. For instance, the vaccine may comprise or encode the mature form of PSCA, the primary translation product, or the full-length translation product of the PSCA gene. In some embodiments, the vaccine comprises the cDNA of PSCA.

In some embodiments, the vaccines, or other compositions of the invention, comprise a polypeptide or comprise a polynucleotide encoding a polypeptide, wherein the polypeptide comprises PSCA. In some embodiments, the polypeptides described herein comprise the mature form of PSCA. In some embodiments, the polypeptides comprise the primary translation product of PSCA. In some embodiments, the polypeptide comprises human PSCA. In some embodiments, the polypeptides described herein comprise the mature form of human PSCA. In some embodiments, the polypeptides comprise the primary translation product of human PSCA.

In addition to the use of naturally occurring forms of PSCA (or polynucleotides encoding those forms), polypeptides comprising fragments of PSCA, or polynucleotides encoding fragments of PSCA may be used in the vaccines. The polypeptides in the vaccines or encoded by polynucleotides of the vaccines are optionally at least about 95%, at least about 90%, at least about 85%, at least about 80%, at least about 75%, at least about 70%, at least about 65%, at least about 60%, at least about 55%, or at least about 50% identical to PSCA.

In some embodiments, the polypeptide does not comprise PSCA, but rather comprises only a fragment of PSCA. In some embodiments, the polypeptides comprise a fragment of PSCA that is at least 8 (consecutive) amino acids in length. Alternatively, in some embodiments, the fragment comprises at least 9 amino acids in length or at least 10 amino acids in length. In some embodiments, the polypeptides comprise a fragment of PSCA that is at least 12, at least 18, at least 26, at least about 50, at least about 75, or at least about 100 amino acids in length. In some embodiments, the polypeptides consist of a fragment of PSCA, wherein the fragment is at least 8, at least 12, at least 18, at least 26, at least about 50, at least about 75, or at least about 100 amino acids in length. In some embodiments, the polypeptides consist of a fragment of PSCA, wherein the fragment is at least 8, at least 12, at least 18, at least 26, at least about 50, or at least about 75, or at least about 100 amino-acids in length. In some embodiments, the fragments of PSCA consist of less than 122, less than 100, less than 75, less than 50, less than 26 contiguous amino acids of the human PSCA sequence. In some embodiments, the fragments of PSCA are not between 8 and 25 amino acids in length.

In some embodiments, the polypeptides comprise a fragment of PSCA, wherein the fragment comprises an MHC class I epitope of PSCA. In some embodiments, the fragment is at least 8 amino acids in length. Alternatively, in some embodiments, the fragment comprises at least 9 amino acids in length or at least 10 amino acids in length. In some embodiments, the polypeptides comprise a fragment of PSCA comprising an MHC class I epitope of PSCA, wherein the fragment is at least 12, at least 18, at least 26, at least about 50, at least about 75, or at least about 100 amino acids in length. In some embodiments, the polypeptides consist of a fragment of PSCA comprising an MHC class I-binding epitope, wherein the fragment is at least 8, at least 12, at least 18, at least 26, at least about 50, at least about 75, or at least about 100 amino acids in length.

In some embodiments, the polypeptides described herein do not comprise, or do not consist of, PSCA (e.g., do not comprise human PSCA). In some embodiments, the polypeptides described herein do not comprise, or do not consist of, the mature form of PSCA. In some embodiments, the polypeptides described herein do not comprise, or do not consist of, the primary translation product of PSCA. In some embodiments, the polypeptides described herein do not comprise, or do not do not consist of, SEQ ID NO:22.

The MHC class I-binding epitopes and the MHC class II-binding epitopes used in vaccines (or other compositions) of the present invention need not necessarily be identical in sequence to the naturally occurring epitope sequences within PSCA. The naturally occurring epitope sequences are not necessarily optimal peptides for stimulating a CTL response. See, for example, (Parkhurst, M. R. et al., J. Immunol., 157:2539-2548, (1996); Rosenberg, S. A. et al., Nat. Med., 4:321-327, (1998)). Thus, there can be utility in modifying an epitope, such that it more readily induces a CTL response. Generally, epitopes may be modified at two types of positions. The epitopes may be modified at amino acid residues that are predicted to interact with the MHC molecule, in which case the goal is to create a peptide sequence that has a higher affinity for the MHC molecule than does the parent epitope. The epitopes can also be modified at amino acid residues that are predicted to interact with the T cell receptor on the CTL, in which case the goal is to create an epitope that has a higher affinity for the T cell receptor than does the parent epitope. Both of these types of modifications can result in a variant epitope that is related to a parent epitope, but which is better able to induce a CTL response than is the parent epitope. In some embodiments, the immunogenicity of the PSCA epitopes may be improved through the optimization of MHC class I processing, MHC class I-binding, and/or T-cell receptor interaction with MHC/peptide complexes. See, e.g., Sette, et al., Tissue Antigens, 59:443-451 (2002), Sette et al., Current Opinion in Immunology, 15:461-470 (2003), and Kersh et al., Nature, 380: 495-8 (1996).

The MHC class I-binding epitopes of PSCA, or the MHC class II-binding epitopes of PSCA identified by application of the methods of the invention can, in some embodiments, be modified by the substitution of one or more residues at different, possibly selective, sites within the epitope sequence. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions are herein defined as exchanges within one of the following five groups: Group 1—small aliphatic, nonpolar or slightly polar residues (Ala, Ser, Thr, Pro, Gly); Group 2—polar, negatively charged residues and their amides (Asp, Asn, Glu, Gln); Group 3—polar, positively charged residues (His, Arg, Lys); Group 4—large, aliphatic, nonpolar residues (Met, Leu, lie, Val, Cys); and Group 4—large, aromatic residues (Phe, Tyr, Trp). An acidic amino acid might also be substituted by a different acidic amino acid or a basic (i.e., alkaline) amino acid by a different basic amino acid. Less conservative substitutions might involve the replacement of one amino acid by another that has similar characteristics but is somewhat different in size, such as replacement of an alanine by an isoleucine residue.

In preferred embodiments, the MHC class I-binding epitope binds to an allelic form of MHC class I that is expressed by the mammal to which the composition is administered or is to be administered.

In some embodiments, the MHC class II-binding epitope binds to an allelic form of MHC class II that is expressed by the mammal to which the composition is administered, or is to be administered.

In some embodiments, the MHC class I-binding epitope is an HLA-A2-restricted epitope, an HLA-A3-restricted epitope, and/or an HLA-A24-restricted epitope. In some embodiments, the MHC class I-binding epitope is an HLA-A2-restricted epitope. In some embodiments, the class I-binding epitope is an HLA-A24-restricted epitope.

In some embodiments, the composition used as a vaccine comprises a polypeptide comprising one or more MHC class I-binding epitopes of PSCA selected from Table 1 of Example 1, below (or a polynucleotide encoding a polypeptide comprising one or more MHC class I-binding epitopes selected from Table 1). In some embodiments, the composition comprises a polypeptide comprising one or more of the epitopes selected from the group consisting of the following peptide #s (see Table 1): 6318; 6319; 6321; 6443; 6444; 6440; and 6441. In some embodiments, the composition comprises a polynucleotide encoding a polypeptide comprising one or more of the epitopes selected from the group consisting of the following peptide #s (see Table 1): 6318; 6319; 6321; 6443; 6444; 6440; and 6441.

In some embodiments, the MHC class I-binding epitope is capable of activating T-lymphocytes from an individual having a successful immunological response to a PSCA-expressing tumor when bound to an HLA molecule on an antigen-presenting cell and contacted with the T-lymphocytes under suitable conditions and for a time sufficient to permit activation of T-lymphocytes.

In some embodiments, the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the epitope is LLPALGLLL (SEQ ID NO:10). In some embodiments, the epitope is YYVGKKNIT (SEQ ID NO:16). In some embodiments, the polypeptide comprises at least two epitopes selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the polypeptide comprises at least three, at least four, at least five, or at least six epitopes selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the polypeptide comprises each of the following epitopes: LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In some alternative embodiments, the MHC class I-binding epitope is not LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO: 15), YYVGKKNIT (SEQ ID NO:16), or ALQPAAAIL (SEQ ID NO:9)

In some embodiments, the fragment of PSCA used in the vaccines, other compositions, and/or methods of the invention comprises an MHC class I-binding epitope selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the fragment comprises LLPALGLLL (SEQ ID NO:10). In some embodiments, the fragment comprises YYVGKKNIT (SEQ ID NO:16). In some embodiments, the fragment comprises at least two, at least three, at least four, at least five, or at least six epitopes selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9). In some embodiments, the fragment comprises each of the following epitopes: LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In some embodiments, the polypeptide comprises an MHC class II-binding epitope. In some embodiments, the polypeptide comprises a plurality of MHC class II-binding epitopes of PSCA. In some embodiments, the polypeptide comprises a plurality of MHC class II-binding epitopes which bind allelic forms of MHC class II that are expressed by the mammal.

In some embodiments, the polypeptide comprises a plurality of MHC class I-binding epitopes. In some embodiments, the polypeptide comprises a plurality of MHC class I-binding epitopes which bind allelic forms of MHC class I that are expressed by the mammal.

In some embodiments, the compositions or vaccines described herein comprise a polypeptide, or a polynucleotide encoding a polypeptide, wherein the polypeptide comprises a fusion protein. The fusion protein comprises a first and a second portion, wherein the first portion comprises an MHC class I-binding epitope of PSCA, and wherein the second portion comprises a sequence of at least 6 amino acid residues, wherein the sequence of the second portion is not in PSCA (or is heterologous to PSCA). In some embodiments, the first portion comprises PSCA. In some embodiments, the PSCA is human PSCA. In some embodiments, the first portion comprises the primary translation product of PSCA. In some embodiments, the first portion comprises mature PSCA. In some embodiments, the first portion comprises, or consists of, a fragment of PSCA. In some embodiments, the fragment of PSCA is at least 8 amino acids in length. In some embodiments, the first portion comprises a fragment of PSCA of at least 10, of at least 12, at least 18, at least 26, at least about 50, at least about 75, or at least about 100 (contiguous) amino acids of PSCA. In certain embodiments, the first portion comprises a fragment of PSCA of at least 10 or at least about 26 amino acids of PSCA. In some embodiments, the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

In some embodiments, the second portion comprises a fragment of at least 8, of at least 12, of at least 16, of at least 20, at least 50, or at least about 100 amino acids in length. In some embodiments, the second portion comprises a signal peptide sequence. In alternative embodiments, the second portion comprises an tag or affinity label. In some embodiments, the second portion is fused to the N-terminus of the first portion. In alternative embodiments, the second portion is fused to the C-terminus of the first portion.

In some embodiments, the vaccines and other compositions of the invention comprise a polypeptide that comprises PSCA. In some embodiments, the polypeptide comprises human PSCA. In some embodiments, the vaccines and other compositions of the invention comprise a polynucleotide encoding a polypeptide that comprises PSCA (e.g., human PSCA).

In some alternative embodiments, the vaccines and other compositions of the invention comprise a polypeptide comprising a variant of PSCA, wherein the variant of PSCA has at least about 70% sequence identity to PSCA, or to a fragment of PSCA that comprises an MHC class I-binding epitope of PSCA. In some embodiments, the variant of PSCA has at least about a 70%, at least about 80%, at least about 90%, at least about 95%, at least about 98%, or at least about 99% sequence identity to PSCA. A “variant of PSCA” or a “PSCA variant” does not encompass PSCA and does not have 100% sequence identity to PSCA. In some other embodiments, the variant of PSCA comprises at least about 70%, at least about 80%, at least about 90%, at least about 95%, at least about 98%, or at least about 99% sequence identity to a fragment of PSCA that comprises an MHC class I-binding epitope of PSCA. In certain embodiments, the fragment of PSCA comprises, or consists of, at least 8, at least 12, at least 18, at least 26, at least about 50, at least about 75, or at least about 100 amino acids of PSCA. In some embodiments, the PSCA is human PSCA. In some embodiments, the PSCA is SEQ ID NO:22.

“Percent (%) sequence identity” (or, alternatively, the “percent (%) identical”), as used herein with respect to amino acid sequences, refers to the percentage of amino acid residues in a candidate sequence (such as a variant of PSCA) that are identical to the amino acid residues in a specific reference sequence (such as in a specific PSCA sequence or in a specific fragment of a PSCA sequence), after aligning the sequences and introducing gaps, if necessary, to achieve the maximum percent sequence identity, and not considering any conservative substitutions a part of the sequence identity. Alignment for purposes of determining percent amino acid sequence identity can be achieved in various ways that are within the skill in the are, for instance, using any of the publicly available algorithms and/or computer software for sequence alignment, or by inspection. Those skilled in the art can determine appropriate parameters for measuring alignment, including any algorithms needed to achieve maximal alignment over the full-length of the sequences being compared. The % sequence identity of a given amino acid sequence A to a given amino acid sequence B is calculated as follows: 100 times the fraction X/Y, where X is the number of identical matches in the optimal alignment of the A and B sequences, and where Y is the total number of amino acid residues in B.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Alternatively, the % (amino acid) sequence identity may be obtained using one of the publicly available BLAST or BLAST-2 programs. The WU-BLAST-2 computer program (Altschul et al., Methods in Enzymology 266:460-480 (1996)). Percent (amino acid) sequence identity may also be determined using the sequence comparison program NCBI-BLAST2 (Altschul et al., Nucleic Acids Res. 25:3389-3402 (1997)). The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997).

The immunogenicity of the compositions described herein, and their ability to induce CD8+ and/or CD4+ T cell responses, can be tested using any one of the techniques known to those of ordinary skill in the art. These methods include standard immunological assays such as ELISPOT assays, Intracellular Cytokine Staining (ICS) assay, cytotoxic T-cell assay, or the like.

In some embodiments, the vaccines and other compositions described herein are acellular. For instance, the composition may be a subunit vaccine or a DNA vaccine. In some embodiments, the composition may comprise a virus.

In some embodiments, the composition comprises a cell, such as an antigen presenting cell (APC) (e.g., a dendritic cell). Antigen presenting cells include such cell types as macrophages, dendritic cells and B cells. Other professional antigen-presenting cells include monocytes, marginal zone Kupffer cells, microglia, Langerhans' cells, interdigitating dendritic cells, follicular dendritic cells, and T cells. Facultative antigen-presenting cells can also be used. Examples of facultative antigen-presenting cells include astrocytes, follicular cells, endothelium and fibroblasts. In some embodiments, the APC has been pulsed or otherwise loaded with a polypeptide comprising an MHC class I epitope of PSCA and/or an MHC class II epitope of PSCA.

In some embodiments, the composition comprises a whole tumor cell. In some embodiments, the composition comprises a whole cell from a tumor cell line that has been selected or modified to overexpress a polypeptide comprising an MHC class I and/or class II epitope relative to the tumor cell line prior to selection or modification. Overexpression can be determined using any of the variety of techniques familiar to one of ordinary skill in the art. For instance, the expression level of a polypeptide in cells can be readily measured at the protein level by Western blot. In some embodiments, the tumor cells that have been selected or modified to overexpress the polypeptide, express the polypeptide at a level that is about 2-fold, at least about 5-fold, at least about 10-fold, or at least about 100-fold greater than cells in the tumor cell line prior to the selection or modification. In some embodiments, the level of expression of the polypeptide in the tumor cell line prior to selection or modification is not detectable.

In some alternative embodiments, the composition does not comprise a whole tumor cell.

In some embodiments, the composition comprises a whole mammalian cell (e.g., a human cell). In some alternative embodiments, the composition does not comprise a whole mammalian cell.

In certain embodiments, the composition comprises a bacterium. In certain embodiments, the composition comprises yeast. In some embodiments, the composition comprises a recombinant vector comprising a bacterium (e.g., Listeria monocytogenes), virus or yeast comprising the polynucleotide and expressing the polypeptide comprising the MHC class I and/or class II epitope.

The compositions described herein can comprise bacterial cells that are transformed to express and/or secrete the polypeptide or to deliver the polynucleotide which is subsequently expressed and/or secreted in cells of the vaccinated individual.

Plasmids and viral vectors, for example, can be used to express a tumor antigen protein in a host cell. The host cell may be any prokaryotic or eukaryotic cell. Thus, for example, a nucleotide sequence derived from the cloning of PSCA polypeptides, encoding all or a selected portion of the full-length protein, can be used to produce a recombinant form of a PSCA polypeptide via microbial or eukaryotic cellular processes. The coding sequence can be ligated into a vector and the loaded vector can be used to transform or transfect hosts, either eukaryotic (e.g., yeast, avian, insect or mammalian) or prokaryotic (bacterial) cells. Such techniques involve standard procedures which are well known in the art.

Typically, expression vectors used for expressing a polypeptide, in vivo or in vitro contain a nucleic acid encoding the desired polypeptide, operably linked to at least one transcriptional regulatory sequence. Regulatory sequences are art-recognized and can be selected to direct expression of the subject proteins in the desired fashion (time and place). Transcriptional regulatory sequences are described, for example, in Goeddel, Gene Expression Technology: Methods in Enzymology 185, Academic Press, San Diego, Calif. (1990).

Accordingly, the present invention provides expression vectors comprising the polynucleotides described herein operably linked to a promoter.

Suitable vectors for the expression of a polypeptide comprising HLA-binding epitopes include plasmids of the types: pBR322-derived plasmids, pEMBL-derived plasmids, pEX-derived plasmids, pBTac-derived plasmids and pUC-derived plasmids for expression in prokaryotic cells, such as E. coli. Mammalian expression vectors may contain both prokaryotic and eukaryotic sequences in order to facilitate the propagation of the vector in bacteria, and one or more eukaryotic transcription units that can be expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. Vaccinia and avian virus vectors can also be used. The methods which may be employed in the preparation of vectors and transformation of host organisms are well known in the art. Other suitable expression systems are well known to those of ordinary skill in the art.

Other types of expression cassettes can also be used. For instance, the references described below in regard to viral, bacterial, and yeast vectors illustrate additional expression vectors which may be used in the present invention.

In another embodiment of the invention, a polypeptide described herein, or a polynucleotide encoding the polypeptide, is delivered to a host organism in an immunogenic composition comprising yeast. The use of live yeast DNA vaccine vectors for antigen delivery has been reviewed recently and reported to be efficacious in a mouse model using whole recombinant Saccharomyces cerevisiae yeast expressing tumor or HIV-1 antigens (see Stubbs et al. (2001) Nature Medicine 7: 625-29).

The use of live yeast vaccine vectors is known in the art. Furthermore, U.S. Pat. No. 5,830,463, the contents of which are incorporated herein by reference, describes particularly useful vectors and systems for use in the instant invention. The use of yeast delivery systems may be particularly effective for use in the tumor/cancer vaccine methods and formulations of the invention as yeast appears to trigger cell-mediated immunity even in the absence of an additional adjuvant. In some embodiments, yeast vaccine delivery systems are nonpathogenic yeast carrying at least one recombinant expression system capable of modulating an immune response.

Bacteria can also be used as carriers for the epitopes of the present invention. Typically the bacteria used are mutant or recombinant. The bacterium is optionally attenuated. For instance, a number of bacterial species have been developed for use as vaccines and can be used in the present invention, including, but not limited to, Shigella flexneri, E. coli, Listeria monocytogenes, Yersinia enterocolitica, Salmonella typhimurium, Salmonella typhi or mycobacterium. The bacterial vector used in the immunogenic composition may be a facultative, intracellular bacterial vector. The bacterium may be used to deliver a polypeptide described herein to antigen-presenting cells in the host organism. The use of live bacterial vaccine vectors for antigen delivery has been reviewed (see, e.g., Medina and Guzman (2001) Vaccine 19: 1573-1580; Weiss and Krusch, (2001) Biol. Chem. 382: 533-41; and Darji et al. (2000) FEMS Immunol and Medical Microbiology 27: 341-9). Furthermore, U.S. Pat. Nos. 6,261,568 and 6,488,926, the contents of which are incorporated herein by reference, describe systems useful for cancer vaccines.

Bacterially mediated gene transfer is particularly useful in genetic vaccination by intramuscular, intradermal, or oral administration of plasmids; such vaccination leads to antigen expression in the vaccinee. Furthermore, in some embodiments, bacteria can provide adjuvant effects and the ability to target inductive sites of the immune system. Furthermore, bacterial vaccine vectors have almost unlimited coding capacity. The use of bacterial carriers is often associated with still other significant benefits, such as the possibility of direct mucosal or oral delivery. Other direct mucosal delivery systems (besides live viral or bacterial vaccine carriers) which can be used include mucosal adjuvants, viral particles, ISCOMs, liposomes, and microparticles.

Microorganisms, including attenuated microorganisms, have been successfully used as carriers for vaccine antigens. In some embodiments, attenuated mucosal pathogens which may be used in the invention include: L. monocytogenes, Salmonella spp., V. cholorae, Shigella spp., mycobacterium, Y. enterocolitica. Commensal strains which can be used in the invention include: S. gordonii, Lactobacillus spp., and Staphylococcus spp. The genetic background of the carrier strain used in the formulation, the type of mutation selected to achieve attenuation, and the intrinsic properties of the immunogen can be adjusted to optimize the extent and quality of the immune response elicited. The general factors to be considered to optimize the immune response stimulated by the bacterial carrier include: selection of the carrier; the specific background strain, the attenuating mutation and the level of attenuation; the stabilization of the attenuated phenotype and the establishment of the optimal dosage. Other antigen-related factors to consider include: intrinsic properties of the antigen; the expression system, antigen-display form and stabilization of the recombinant phenotype; co-expression of modulating molecules and vaccination schedules.

Salmonella typhimurium can be used as a bacterial vector in the immunogenic compositions of the invention. Use of this bacterium as an effective vector for a vaccine has been demonstrated in the art. For instance, the use of S. typhimurium as an attenuated vector for oral somatic transgene vaccination has been described (see, e.g., Darji et al. (1997) Cell 91: 765-775; and Darji et al. (2000) FEMS Immun and Medical Microbiology 27: 341-9). Indeed most knowledge of bacteria-mediated gene transfer has been acquired using attenuated S. typhimurium as carrier. Two metabolically attenuated strains that have been used include S. typhimurium aroA, which is unable to synthesize aromatic amino acids, and S. typhimurium 22-11, which is defective in purine metabolism. Several antigens have been expressed using these carriers: originally, listeriolysin and actA (two virulence factors of L. monocytogenes) and beta-galactosidase (β-gal) of E. coli were successfully tested. Cytotoxic and helper T cells as well as specific antibodies could be detected against these antigens following oral application of a single dose of the recombinant salmonella. In addition, immunization with Salmonella carrying a listeriolysin-encoding expression plasmid elicited a protective response against a lethal challenge with L. monocytogenes. Oral transgene vaccination methodology has now been extended to include protective responses in herpes simplex virus 2 and hepatitis B infection models, with cell-mediated immune responses detected at the mucosal level.

In tumor models using β-gal as a surrogate tumor antigen, partial protective immunity against an aggressive fibrosarcoma was induced by orally administering Salmonella carrying a β-gal-encoding plasmid (see Paglia et al. (1998) Blood 92: 3172-76). In similar experiments using a β-gal-expressing transfectant of the murine renal cell carcinoma line RENCA, Zller and Christ (Woo et al. (2001) Vaccine 19: 2945-2954) demonstrated superior efficacy when the antigen-encoding plasmid was delivered in bacterial carriers as opposed to using naked DNA. Interestingly, Salmonella can be used to induce a tumor growth retarding response against the murine melanoma B16; the Salmonella carry minigenes encoding epitopes of the autologous tumor antigens gp100 and TRP2 fused to ubiquitin. This suggests that under such circumstances peripheral tolerance towards autologous antigens can be overcome. This was confirmed by the same group (Lode et al. (2000) Med Ped Oncol 35: 641-646 using similar constructs of epitopes of tyrosine hydroxylase as autologous antigen in a murine neuroblastoma system. Furthermore, these findings were recently extended by immunizing mice that were transgenic for human carcinogenic antigen (hCEA) using a plasmid encoding a membrane-bound form of complete hCEA. In this case, a hCEA-expressing colon carcinoma system was tested and protection against a lethal challenge with the tumor could be improved by systemic application of interleukin 2 (IL-2) as adjuvant during the effector phase (see Xiang et al. (2001) Clin Cancer Res 7: 856s-864s).

Another bacterial vector which may be used in the immunogenic compositions described herein is Salmonella typhi. The S. typhi strain commonly used for immunization—Ty21a galE—lacks an essential component for cell-wall synthesis. Recently developed improved strains include those attenuated by a mutation in guaBA, which encodes an essential enzyme of the guanine biosynthesis pathway (Pasetti et al., Infect. Immun. (2002) 70:4009-18; Wang et al., Infect. Immun. (2001) 69:4734-41; Pasetti et al., Clin. Immunol. (1999) 92:76-89). Additional references describing the use of Salmonella typhi and/or other Salmonella strains as delivery vectors for DNA vaccines include the following: Lundin, Infect. Immun. (2002) 70:5622-7; Devico et al., Vaccine, (2002) 20:1968-74; Weiss et al., Biol. Chem. (2001) 382:533-41; and Bumann et al., FEMS Immunol. Med. Microbiol. (2000) 27:357-64.

The vaccines and immunogenic compositions of the present invention can employ Shigella flexneri as a delivery vehicle. S. flexneri represents the prototype of a bacterial DNA transfer vehicle as it escapes from the vacuole into the cytosol of the host cell. Several attenuated mutants of S. flexneri have been used successfully to transfer DNA to cell lines in vitro. Auxotrophic strains were defective in cell-wall synthesis (Sizemore et al. (1995) Science 270: 299-302 and Courvalin et al. (1995) C R Acad Sci Ser III, 318: 1207-12), synthesis of aromatic amino acids (Powell et al. (1996) Vaccines 96: Molecular Approaches to the Control of Infectious Disease; Cold Spring Harbor Laboratory Press) or synthesis of guanine nucleotides (Anderson et al. (2000) Vaccine 18: 2193-2202).

In some embodiments, the vaccines and immunogenic compositions of the present invention comprise Listeria monocytogenes (Portnoy et al, Journal of Cell Biology, 158:409-414 (2002); Glomski et al., Journal of Cell Biology, 156:1029-1038 (2002)). The ability of L. monocytogenes to serve as a vaccine vector has been reviewed in Wesikirch, et al., Immunol. Rev. 158:159-169 (1997). Strains of Listeria monocytogenes have recently been developed as effective intracellular delivery vehicles of heterologous proteins providing delivery of antigens to the immune system to induce an immune response to clinical conditions that do not permit injection of the disease-causing agent, such as cancer (U.S. Pat. No. 6,051,237; Gunn et al., J. Of Immunology, 167:6471-6479 (2001); Liau, et al., Cancer Research, 62: 2287-2293 (2002); U.S. Pat. No. 6,099,848; WO 99/25376; and WO 96/14087) and HIV (U.S. Pat. No. 5,830,702). A recombinant L. monocytogenes vaccine expressing an lymphocytic choriomeningitis virus (LCMV) antigen has also been shown to induce protective cell-mediated immunity to the antigen (Shen et al., Proc. Natl. Acad. Sci. USA, 92: 3987-3991 (1995).

As a facultative intracellular bacterium, L. monocytogenes elicits both humoral and cell-mediated immune responses. Following entry of Listeria into a cell of the host organism, the Listeria produces Listeria-specific proteins that enable it to escape from the phagolysosome of the engulfing host cell into the cytosol of that cell. Here, L. monocytogenes proliferates, expressing proteins necessary for survival, but also expressing heterologous genes operably linked to Listeria promoters. Presentation of peptides of these heterologous proteins on the surface of the engulfing cell by MHC proteins permit the development of a T cell response. Two integration vectors that are useful for introducing heterologous genes into the bacteria for use as vaccines include pL1 and pL2 as described in Lauer et al., Journal of Bacteriology, 184: 4177-4186 (2002).

In addition, attenuated forms of L. monocytogenes useful in immunogenic compositions have been produced. The ActA protein of L. monocytogenes is sufficient to promote the actin recruitment and polymerization events responsible for intracellular movement. A human safety study has reported that oral administration of an actA/plcB-deleted attenuated form of Listeria monocytogenes caused no serious sequelae in adults (Angelakopoulos et al., Infection and Immunity, 70:3592-3601 (2002)). Other types of attenuated forms of L. monocytogenes have also been described (see, for example, WO 99/25376 and U.S. Pat. No. 6,099,848, which describe auxotrophic, attenuated strains of Listeria that express heterologous antigens). Additional attenuated forms of Listeria monocytogenes which can express heterologous antigens and be used as recombinant vectors in vaccines are described in, for example, U.S. Publication Nos. 2004/0228877, 2004/0197343, 2005/0249748, and 2005/0281783, each of which is hereby incorporated by reference herein in its entirety.

Yersinia enterocolitica is another intracellular bacterium that can optionally be used as a bacterial vector in immunogenic compositions of the present invention. The use of attenuated strains of Yersini enterocolitica as vaccine vectors is described in PCT Publication No. WO 02/077249.

In further embodiments of the invention, the immunogenic compositions of the invention comprise mycobacterium, such as Bacillus Calmette-Guerin (BCG). The Bacillus of Calmette and Guerin has been used as a vaccine vector in mouse models (Gicquel et al., Dev. Biol. Stand 82:171-8 (1994)). See also, Stover et al., Nature 351: 456-460 (1991).

Alternatively, viral vectors can be used. The viral vector will typically comprise a highly attenuated, non-replicative virus. Viral vectors include, but are not limited to, DNA viral vectors such as those based on adenoviruses, herpes simplex virus, avian viruses, such as Newcastle disease virus, poxviruses such as vaccinia virus, and parvoviruses, including adeno-associated virus; and RNA viral vectors, including, but not limited to, the retroviral vectors. Vaccinia vectors and methods useful in immunization protocols are described in U.S. Pat. No. 4,722,848. Retroviral vectors include murine leukemia virus, and lentiviruses such as human immunodeficiency virus. Naldini et al. (1996) Science 272:263-267. Replication-defective retroviral vectors harboring a polynucleotide of the invention as part of the retroviral genome can be used. Such vectors have been described in detail. (Miller, et al. (1990) Mol. Cell Biol. 10:4239; Kolberg, R. (1992) J. NIH Res. 4:43; Cornetta, et al. (1991) Hum. Gene Therapy 2:215).

Adenovirus and adeno-associated virus vectors useful in this invention may be produced according to methods already taught in the art. (See, e.g., Karlsson, et al. (1986) EMBO 5:2377; Carter (1992) Current Opinion in Biotechnology 3:533-539; Muzcyzka (1992) Current Top. Microbiol. Immunol. 158:97-129; Gene Targeting: A Practical Approach (1992) ed. A. L. Joyner, Oxford University Press, NY). Several different approaches are feasible.

Alpha virus vectors, such as Venezuelan Equine Encephalitis (VEE) virus, Semliki Forest virus (SFV) and Sindbis virus vectors, can be used for efficient gene delivery. Replication-deficient vectors are available. Such vectors can be administered through any of a variety of means known in the art, such as, for example, intranasally or intratumorally. See Lundstrom, Curr. Gene Ther. 2001 1:19-29.

Additional references describing viral vectors which could be used in the methods of the present invention include the following: Horwitz, M. S., Adenoviridae and Their Replication, in Fields, B., et al. (eds.) Virology, Vol. 2, Raven Press New York, pp. 1679-1721, 1990); Graham, F. et al., pp. 109-128 in Methods in Molecular Biology, Vol. 7: Gene Transfer and Expression Protocols, Murray, E. (ed.), Humana Press, Clifton, N.J. (1991); Miller, et al. (1995) FASEB Journal 9:190-199, Schreier (1994) Pharmaceutica Acta Helvetiae 68:145-159; Schneider and French (1993) Circulation 88:1937-1942; Curiel, et al. (1992) Human Gene Therapy 3:147-154; WO 95/00655; WO 95/16772; WO 95/23867; WO 94/26914; WO 95/02697 (Jan. 26, 1995); and WO 95/25071.

In another form of vaccine, DNA is complexed with liposomes or ligands that often target cell surface receptors. The complex is useful in that it helps protect DNA from degradation and helps target plasmid to specific tissues. The complexes are typically injected intravenously or intramuscularly.

Polynucleotides used as vaccines can be used in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g., with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647.

In addition, complex coacervation is a process of spontaneous phase separation that occurs when two oppositely charged polyelectrolytes are mixed in an aqueous solution. The electrostatic interaction between the two species of macromolecules results in the separation of a coacervate (polymer-rich phase) from the supernatant (polymer-poor phase). This phenomenon can be used to form microspheres and encapsulate a variety of compounds. The encapsulation process can be performed entirely in aqueous solution and at low temperatures, and has a good chance, therefore, of preserving the bioactivity of the encapsulant. In developing an injectable controlled release system, the complex coacervation of gelatin and chondroitin sulfate to encapsulate a number of drugs and proteins has been exploited (see Truong, et al. (1995) Drug Delivery 2: 166) and cytokines have been encapsulated in these microspheres for cancer vaccination (see Golumbek et al. (1993) Cancer Res 53: 5841). Anti-inflammatory drugs have also been incorporated for intra-articular delivery to the joints for treating osteoarthritis (Brown et al. (1994) 331: 290). U.S. Pat. Nos. 6,193,970, 5,861,159 and 5,759,582, describe compositions and methods of use of complex coacervates for use as DNA vaccine delivery systems of the instant invention. In particular, U.S. Pat. No. 6,475,995, teaches DNA vaccine delivery systems utilizing nanoparticle coacervates of nucleic acids and polycations which serve as effective vaccines when administered orally.

The present invention provides a variety of immunogenic compositions, including vaccines, that are capable of inducing an antitumor immune response in a mammal. The induced immune response is optionally a cell-mediated immune response, a humoral immune response, or both.

In certain embodiments, the antitumor immune response that is induced comprises a T-cell response to PSCA. In some embodiments, the immune response is a T-cell response that comprises induction of PSCA specific CD8+ T cells and/or PSCA specific CD4+ T cells. In some embodiments, the vaccine or composition comprises a polypeptide comprising an MHC class I-binding epitope (or a polynucleotide encoding such a polypeptide) and the T-cell response induced by administration of the vaccine or composition to a mammal is a PSCA-specific CD8+ T cell response. In some embodiments, the vaccine or composition comprises a polypeptide comprising an MHC class II-binding epitope (or a polynucleotide encoding such a polypeptide) and the T-cell response induced by administration of the vaccine or composition to a mammal is a PSCA-specific CD4+ T cell response. In certain embodiments, both PSCA-specific CD4+ and PSCA-specific CD8+ T cell responses are induced.

In some embodiments, the compositions described herein are immunogenic. In some embodiments, the immunogenic compositions are useful as vaccines for the treatment of cancer. In some embodiments, the compositions are useful as prophylactic vaccines. In some alternative embodiments, the vaccines are useful as therapeutic vaccines. In some embodiments, the compositions described herein are pharmaceutical compositions.

In some embodiments, the composition is administered in an amount sufficient to induce tumor regression or inhibit progression of a cancer in the mammal. In some embodiments, the composition is administered in an amount sufficient to delay or prevent recurrence of cancer in the mammal, wherein the mammal has had the tumor removed.

The positive effects of treatment of cancer with the compositions described herein may include, but are not necessarily limited to, one or more of the following positive effects: induction of tumor regression, inhibition of progression of a cancer, inhibition of recurrence of cancer, decrease in pain associated with the cancer, and/or increased survivability. In certain embodiments, the treatment of cancer involves the eradication, reduction, or amelioration of one or more symptoms associated with cancer. In certain embodiments, the positive effects of treatment of cancer include, but are not necessarily limited to, reducing the proliferation of (or destroying) neoplastic or cancerous cells, reducing metastasis of neoplastic cells found in cancers, shrinking the size of a tumor, increasing the quality of life of a subject suffering from the cancer, and/or decreasing the dose of other medications required to treat the disease.

In other embodiments, the mammal is murine or primate. In some embodiments, the mammal is a human. In some embodiments, the mammal is a rat, mouse, ape, rabbit, or guinea pig.

In certain embodiments, the mammal is a mammal (e.g., a human) that has a tumor that expresses PSCA, or that has had a tumor that expresses PSCA removed. In certain embodiments, the mammal is a mammal at risk for developing a tumor that expresses PSCA. In some embodiments, the tumor overexpresses PSCA relative to the normal tissue from which the tumor is derived.

The immune responses induced by the compositions of the invention can be measured by both in vitro and in vivo methods to determine if the composition is effective.

The efficacy of all of the compositions described herein can be also evaluated in animal models, such as a mouse models. One established animal model for human prostate cancer is the transgenic adenocarcinoma of the mouse prostate (TRAMP) (see, e.g., Ross et al., American Journal of Pathology, 158: 809-816 (2001); Yang et al., Cancer Research, 61:5857-5860 (2001); Drake et al., Cancer Cell, 7:239-249 (2005)). In addition, various human prostate and pancreatic cancer xenograft mouse models have been successfully used to test the efficacy of anti-PSCA therapeutic antibodies and immunoconjugates (see, e.g., Gu et al., Cancer Res., 65: 9495-9500 (2005); Saffran et al., PNAS, 98:2658-2663); Ross et al., Cancer Research 62:2546-2553 (2002); and Wente et al., Pancreas 31:119-125 (2005)).

By way of non-limiting example, to test candidate cancer vaccines in a mouse model, the candidate vaccine containing the desired tumor antigen can be administered to a population of mice either before or after challenge with a tumor cell line expressing PSCA. Thus, a mouse model can be used to test for both therapeutic and prophylactic effects of a candidate vaccine. Vaccination with a candidate vaccine can be compared to control populations that are either not vaccinated, vaccinated with vehicle alone, or vaccinated with a vaccine that comprises an irrelevant antigen. If the vaccine is a recombinant microbe, for example, its relative efficacy can be compared to a population of microbes in which the genome has not been modified to express the antigen. The effectiveness of a candidate vaccine can be evaluated, e.g., in terms of effect on tumor volume or in terms of survival rates. The tumor volume in mice vaccinated with candidate vaccine may be about 5%, about 10%, about 25%, about 50%, about 75%, about 90% or about 100% less than the tumor volume in mice that are either not vaccinated or are vaccinated with vehicle or a vaccine that expresses (or otherwise comprises) an irrevelant antigen. The differential in tumor volume may be observed at least about 10, at least about 17, or at least about 24 days following the implantation of the tumor cells into the mice. The median survival time in mice vaccinated with a nucleic acid-modified microbe may be, for example, at least about 2, at least about 5, at least about 7, or at least about 10 days longer than in mice that are either not vaccinated or are vaccinated with vehicle or a vaccine that comprises an irrelevant antigen.

The vaccines of the present invention can be administered by any means known in the art for inducing a T cell cytolytic response. These means include oral administration, intravenous injection, percutaneous scarification, subcutaneous injection, intramuscular injection, and intranasal administration. The vaccines can be administered intradermally by gene gun. Gold particles coated with DNA may be used in the gene gun. Other inoculation routes as are known in the art can be used.

In some embodiments, the vaccine and other compositions described herein further comprise a non-PSCA antigen. In some embodiments, the non-PSCA antigen is a tumor-associated antigen or is derived from a tumor-associated antigen.

In some embodiments, the vaccines and other compositions described herein are administered to a mammal, wherein the mammal is further treated with chemotherapy, radiation, surgery, or hormone therapy. In some embodiments, the mammal is further treated with additional immunotherapy.

In some embodiments, the vaccines and other compositions described herein comprise an adjuvant. As used herein, an adjuvant increases the ability of the PSCA antigen to stimulate the immune system. Adjuvants include, without limitation, B7 costimulatory molecule, interleukin-2, interferon-gamma, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants which stimulate a cytolytic T cell response versus an antibody response are preferred, although those that stimulate both types of response can be used as well. In some embodiments, adjuvants such as aluminum hydroxide or aluminum phosphate, are added to increase the ability of the vaccine to trigger, enhance, or prolong an immune response. Additional materials, such as cytokines, chemokines, and bacterial nucleic acid sequences, like CpG, are also potential adjuvants. Other representative examples of adjuvants include the synthetic adjuvant QS-21 comprising a homogeneous saponin purified from the bark of Quillaja saponaria and Corynebacterium parvum (McCune et al., Cancer, 1979; 43:1619). It will be understood that the adjuvant is subject to optimization. In other words, the skilled artisan can engage in routine experimentation to determine the best adjuvant to use.

The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) which can be used in the preparation of unit dosage forms.

A reagent used in therapeutic methods of the invention is typically present in a pharmaceutical composition. Pharmaceutical compositions typically comprise a pharmaceutically acceptable carrier, which meets industry standards for sterility, isotonicity, stability, and non-pyrogenicity and which is nontoxic to the recipient at the dosages and concentrations employed. The particular carrier used depends on the type and concentration of the therapeutic agent in the composition and the intended route of administration. If desired, a stabilizing compound can be included. Formulation of pharmaceutical compositions is well known and is described, for example, in U.S. Pat. Nos. 5,580,561 and 5,891,725.

In certain embodiments, the compositions of the invention include pharmaceutical compositions comprising the polypeptides or polynucleotides described herein. In some embodiments, the compositions further comprise a pharmaceutically acceptable carrier.

As used herein, “carrier” includes any and all solvents, dispersion media, vehicles, coatings, diluents, antifungal agents, isotonic and absorption delaying agents, buffers, carrier solutions, suspensions, colloids, and the like. Pharmaceutically acceptable carriers are well known to those of ordinary skill in the art, and include any material which, when combined with an active ingredient, allows the ingredient to retain biological activity and is non-reactive with the subject's immune system. For instance, pharmaceutically acceptable carriers include, but are not limited to, water, buffered saline solutions (e.g., 0.9% saline), emulsions such as oil/water emulsions, and various types of wetting agents. Possible carriers also include, but are not limited to, oils (e.g., mineral oil), dextrose solutions, glycerol solutions, chalk, starch, salts, glycerol, and gelatin.

While any suitable carrier known to those of ordinary skill in the art may be employed in the pharmaceutical compositions, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. In some embodiments, for parenteral administration, such as subcutaneous injection, the carrier comprises water, saline, alcohol, a fat, a wax or a buffer. In some embodiments, any of the above carriers or a solid carrier, such as mannitol, lactose, starch, magnesium stearate, sodium saccharine, talcum, cellulose, glucose, sucrose, and magnesium carbonate, are employed for oral administration.

Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Further additives, such as preservatives, stabilizers, adjuvants, antibiotics, and other substances can be used as well. Preservatives, such as thimerosal or 2-phenoxy ethanol, can be added to slow or stop the growth of bacteria or fungi resulting from inadvertent contamination, especially as might occur with vaccine vials intended for multiple uses or doses. Stabilizers, such as lactose or monosodium glutamate (MSG), can be added to stabilize the vaccine formulation against a variety of conditions, such as temperature variations or a freeze-drying process.

Viral vectors can be used to administer polynucleotides encoding a polypeptide comprising a PSCA epitope. Such viral vectors include vaccinia virus and avian viruses, such as Newcastle disease virus. Others may be used as are known in the art.

One particular method for administering polypeptide vaccine is by pulsing the polypeptide onto an APC or dendritic cell in vitro. The polypeptide binds to MHC molecules on the surface of the APC or dendritic cell. Prior treatment of the APCs or dendritic cells with interferon-gamma. can be used to increase the number of MHC molecules on the APCs or dendritic cells. The pulsed cells can then be administered as a carrier for the polypeptide. Peptide pulsing is taught in Melero et al., Gene Therapy 7:1167 (2000).

Naked DNA can be injected directly into the host to produce an immune response. Such naked DNA vaccines may be injected intramuscularly into human muscle tissue, or through transdermal or intradermal delivery of the vaccine DNA, typically using biolistic-mediate gene transfer (i.e., gene gun). Recent reviews describing the gene gun and muscle injection delivery strategies for DNA immunization include Tuting, Curr. Opin. Mol. Ther. (1999) 1: 216-25, Robinson, Int. J. Mol. Med. (1999) 4: 549-55, and Mumper and Ledbur, Mol. Biotechnol. (2001) 19: 79-95. Other possible methods for delivering plasmid DNA includes electroporation and iontophoreses.

Another possible gene delivery system comprises ionic complexes formed between DNA and polycationic liposomes (see, e.g., Caplen et al. (1995) Nature Med. 1: 39). Held together by electrostatic interaction, these complexes may dissociate because of the charge screening effect of the polyelectrolytes in the biological fluid. A strongly basic lipid composition can stabilize the complex, but such lipids may be cytotoxic. Other possible methods for delivering DNA include electroporation and iontophoreses.

The use of intracellular and intercellular targeting strategies in DNA vaccines may further enhance the PSCA-specific antitumor effect. Previously, intracellular targeting strategies and intercellular spreading strategies have been used to enhance MHC class I or MHC class II presentation of antigen, resulting in potent CD8+ or CD4+ T cell-mediated antitumor immunity, respectively. For example, MHC class I presentation of a model antigen, HPV-16 E7, was enhanced using linkage of Mycobacterium tuberculosis heat shock protein 70 (HSP70) (Chen, et al., (2000), Cancer Research, 60: 1035-1042), calreticulin (Cheng, et al., (2001) J Clin Invest, 108:669-678) or the translocation domain (domain II) of Pseudomonas aeruginosa exotoxin A (ETA(dII)) (Hung, et al., (2001) Cancer Research, 61: 3698-3703) to E7 in the context of a DNA vaccine. To enhance MHC class II antigen processing, the sorting signals of the lysosome associated membrane protein (LAMP-1) have been linked to the E7 antigen, creating the Sig/E7/LAMP-1 chimera (Ji, et al, (1999), Human Gene Therapy, 10: 2727-2740). To enhance further the potency of naked DNA vaccines, an intercellular strategy that facilitates the spread of antigen between cells can be used. This improves the potency of DNA vaccines as has been shown using herpes simplex virus (HSV-1) VP22, an HSV-1 tegument protein that has demonstrated the remarkable property of intercellular transport and is capable of distributing protein to many surrounding cells (Elliot, et al., (1997) Cell, 88: 223-233). Such enhanced intercellular spreading of linked protein, results in enhancement of antigen-specific CD8+ T cell-mediated immune responses and antitumor effect. Any such methods can be used to enhance DNA vaccine potency against PSCA-expressing tumors.

The vaccines, polynucleotides, polypeptides, cells, and viruses of the present invention can be administered to either human or other mammals. The other mammals can be domestic animals, such as goats, pigs, cows, horses, and sheep, or can be pets, such as dogs, rabbits, and cats. The other mammals can be experimental subjects, such as mice, rats, rabbits, monkeys, or donkeys.

In preferred embodiments of the methods of the invention described herein, such as the methods of inducing a T-cell response to tumors and methods of treating cancer, the methods comprise the administration of an effective amount of a composition or vaccine described herein to the mammal (e.g., human). In some embodiments, an effective amount is the amount necessary to induce a T-cell response to a tumor (e.g., a PSCA-specific CD8+ T cell response) in the mammal to which the composition is administered. In some embodiments, an effective amount is the amount necessary to treat cancer in the mammal to which the composition is delivered.

The determination of a therapeutically effective dose is well within the capability of those skilled in the art. A therapeutically effective dose refers to that amount of active ingredient that increases anti-tumor cytolytic T-cell activity relative to that which occurs in the absence of the therapeutically effective dose.

For any substance, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

Pharmaceutical compositions that exhibit large therapeutic indices are preferred. The data obtained from cell culture assays and animal studies are used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

Effective doses may be extrapolated from dose-response curves derived from animal model test systems. In certain embodimenst, the dosage ranges are 0.0001-fold to 10,000-fold of the murine LD50, 0.01-fold to 1,000-fold of the murine LD50, 0.1-fold to 500-fold of the murine LD50, 0.5-fold to 250-fold of the murine LD50, 1-fold to 100-fold of the murine LD50, and 5-50-fold of the murine LD50.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Effective in vivo dosages of polynucleotides and polypeptides are in the range of about 100 ng to about 200 ng, 500 ng to about 50 mg, about 1 μg to about 2 mg, about 5 μg to about 500 μg, and about 20 μg to about 100 μg.

In certain embodiments, where the composition comprises a bacterial vector, the dosage may be based on the amount of colony forming units (CFU) or on the number of organisms. In some embodiments, a single dose of the composition comprises at least about 1×10⁵ organisms, at least about 1×10⁶ organisms, or at least about 1×10⁷ organisms. In some embodiments, the composition comprises from about 1 CFU/kg to about 1×10¹⁰ CFU/kg, from about 10 CFU/kg to about 1×10⁹ CFU/kg, from about 1×10² CFU/kg to about 1×10⁸ CFU/kg, from about 1×10³ CFU/kg to about 1×10⁸ CFU/kg, or from about 1×10⁴ CFU/kg to about 1×10⁷ CFU/kg.

Desirable immunogens for use as anti-tumor vaccines are those which are highly differentially expressed between tumors and their corresponding normal tissues. Expression differences are preferably at least 2-fold, 3-fold, 4-fold, 5-fold, or even 10 fold. Expression can be measured by any means known in the art, including but not limited to SAGE, microarrays, Northern blots, and Western blots. Interest in such proteins as immunogens is enhanced by determining that humans respond to immunization with the protein (or gene encoding it) by generating CD4 or CD8 T cells which are specifically activated by the protein. Testing for such activation can be done, inter alia, using TAP deficient cell lines such as the human T2 cell line to present potential antigens in an MHC complex. Activation can be measured by any assay known in the art. One such assay is the ELISPOT assay.

Future responses to tumor vaccines can be predicted based on the response of CD8+ and or CD4+ T cells. If the tumor vaccine comprises PSCA or at least one T cell epitope, then monitoring of the CD8+ and or CD4+ response to PSCA provides useful prognostic information. A robust CD8+ and or CD4+ response indicates that the patient has mounted an effective immunological response and will survive significantly longer than those who have not mounted such a response. The tumor vaccine may comprise whole tumor cells, particularly pancreatic, prostate, or bladder tumor cells. The tumor vaccine may comprise a polyethylene glycol fusion of tumor cells and dendritic cells. The tumor vaccine may comprise apoptotic or necrotic tumor cells which have been incubated with dendritic cells. The tumor vaccine may comprise mRNA or whole RNA which has been incubated with dendritic cells. The T cell responses to PSCA can be measured by any assay known in the art, including an ELISPOT assay. Alternatively, future response to such a tumor vaccine can be monitored by assaying for a delayed type hypersensitivity respone to PSCA. Such a response has been identified as a positive prognostic indicator.

### EXAMPLES

### Example 1

To identify genes that can serve as immune targets for the majority of pancreatic adenocarcinoma patients, only those genes that were non-mutated, overexpressed by the majority of pancreatic cancer patients, and thought to be overexpressed by the vaccine cell lines, were focused on. One of these genes was PSCA.

A combination of two public use computer algorithms were used to predict peptide nonamers that bind to three common human leukocyte antigen (HLA)-class I molecules. The predictive algorithm “BIMAS”, ranks potential HLA binding epitopes according to the predictive half-time disassociation of peptide/HLA complexes. The “SYFPEITHI” algorithm ranks peptides according to a score that accounts for the presence of primary and secondary HLA-binding anchor residues. Both computerized algorithms score candidate epitopes based on amino acid sequences within a given protein that have similar binding motifs to previously published HLA binding epitopes.

PSCA peptides predicted to bind HLA-A2, A3, and A24 are listed in Table 1, below.

TABLE 1
PSCA Peptides Predicted to Bind to HLA-A2, A3, and A24

| Peptide | HLA- | AminoAcid | | |
|---|---|---|---|---|
| # | Restriction | Sequence | Amino Acid Position in Protein | |
| 6318 | HLA-A2 | LLALLMAGL | PSCA₍₅₋₁₃₎ | (SEQ ID NO: 5) |
| 6319 | HLA-A2 | ALQPGTALL | PSCA₍₁₄₋₂₂₎ | (SEQ ID NO: 6) |
| 6320 | HLA-A2 | ALLPALGLL | PSCA₍₁₀₈₋₁₁₆₎ | (SEQ ID NO: 7) |
| 6321 | HLA-A2 | ALLMAGLAL | PSCA₍₇₋₁₅₎ | (SEQ ID NO: 8) |
| 6443 | HLA-A2 | ALQPAAAIL | PSCA₍₉₉₋₁₀₇₎ | (SEQ ID NO: 9) |
| 6444 | HLA-A2 | LLPALGLLL | PSCA₍₁₀₉₋₁₁₇₎ | (SEQ ID NO: 10) |
| 6445 | HLA-A2 | QLGEQCWTA | PSCA₍₄₃₋₅₁₎ | (SEQ ID NO: 11) |
| 6446 | HLA-A2 | ALLCYSCKA | PSCA₍₂₀₋₂₈₎ | (SEQ ID NO: 12) |
| 6447 | HLA-A2 | AILALLPAL | PSCA₍₁₀₅₋₁₁₃₎ | (SEQ ID NO: 13) |
| 6318 | HLA-A3 | LLALLMAGL | PSCA₍₅₋₁₃₎ | (SEQ ID NO: 5) |
| 6319 | HLA-A3 | ALQPGTALL | PSCA₍₁₄₋₂₂₎ | (SEQ ID NO: 6) |
| 6321 | HLA-A3 | ALLMAGLAL | PSCA₍₇₋₁₅₎ | (SEQ ID NO: 8) |
| 6322 | HLA-A3 | RIRAVGLLT | PSCA₍₅₂₋₆₀₎ | (SEQ ID NO: 14) |
| 6443 | HLA-A3 | ALQPAAAIL | PSCA₍₉₉₋₁₀₇₎ | (SEQ ID NO: 9) |
| 6444 | HLA-A3 | LLPALGLLL | PSCA₍₁₀₉₋₁₁₇₎ | (SEQ ID NO: 10) |
| 6445 | HLA-A3 | QLGEQCWTA | PSCA₍₄₃₋₅₁₎ | (SEQ ID NO: 11) |
| 6446 | HLA-A3 | ALLCYSCKA | PSCA₍₂₀₋₂₈₎ | (SEQ ID NO: 12) |
| 6320 | HLA-A24 | ALLPALGLL | PSCA₍₁₀₈₋₁₁₆₎ | (SEQ ID NO: 7) |
| 6440 | HLA-A24 | DYYVGKKNI | PSCA₍₇₆₋₈₄₎ | (SEQ ID NO: 15) |
| 6441 | HLA-A24 | YYVGKKNIT | PSCA₍₇₇₋₈₅₎ | (SEQ ID NO: 16) |
| 6443 | HLA-A24 | ALQPAAAIL | PSCA₍₉₉₋₁₀₇₎ | (SEQ ID NO: 9) |
| 6444 | HLA-A24 | LLPALGLLL | PSCA₍₁₀₉₋₁₁₇₎ | (SEQ ID NO: 10) |

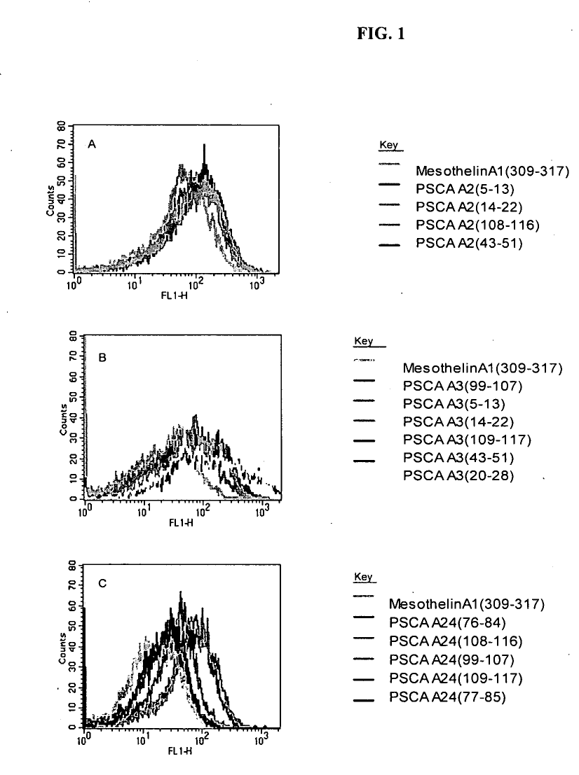

Binding of the epitopes to their respective HLA class I molecule was tested by pulsing TAP deficient T2 cells that expressed the corresponding HLA class I molecule (T2-A2, T2-A3, or T2-A24). The results of T2 binding experiments identifying PSCA derived peptides that bind to HLA-A2, HLA-A3, and HLA-A24 molecules is shown in FIG. 1A-1C.

Materials and Methods. Identification of candidate genes and epitope selection. Serial Analysis of Gene Expression (SAGE) was used to identify prostate stem cell antigen (PSCA) as one of the genes overexpressed in pancreatic cancer cell lines and fresh tissue. Two computer algorithms that are available to the general public and accessible through the internet were used to predict PSCA-derived peptides that bind to HLA-A2, A3, and A24 molecules. “BIMAS” was developed by K. C. Parker and collaborators (bimas.dcrt.nih.gov) and “SYFPEITHI” was developed by Rammensee et al. (www.uni-tuebingen.de/uni/kxi).

Materials and Methods. Peptides and T2 cell lines. All peptides were purified to greater than 95% purity and synthesized by Macromolecular Resources (Fort Collins, Colo.) according to published sequences: M1 peptide GILGFVFTL (SEQ ID NO:17), derived from influenza matrix protein (amino acid positions 58-66), PSCA A2 peptides that were identified using the available databases and HIV-gag A2 peptide SLYNTVATL (amino acid positions 75-83) (SEQ ID NO:2) contain an HLA-A2 binding motif. PSCA A3 peptides, HIV-NEF A3 peptide QVPLRPMTYK (SEQ ID NO:3) (amino acid positions 94-103), and Flu NP peptide ILRGSVAHK (SEQ ID NO:18), derived from Influenza A (PR8) Nucleoprotein (amino acid positions 265-273) contain an HLA-A3 binding motif. PSCA A24 peptides, Tyrosinase peptide AFLPWHRLF (amino acid positions 206-214) (SEQ ID NO:4), and EBV EBNA3C peptide RYEDPDAPL (amino acid positions 721-729) (SEQ ID NO:19) contain an HLA-A24 binding motif. Stock solutions (1-2 mg/ml) of PSCA and control peptides were prepared in 10-100% DMSO (JTBaker, Phillippsburg, N.J.) and further diluted in cell culture medium for each assay. The T2 cells are a human B and T lymphoblast hybrid that only expresses the HLA-A*0201 allele. T2 cells are TAP deficient and therefore fail to transport newly processed HLA class I-binding epitopes from the cytosol into the endoplasmic reticulum where these epitopes would normally bind to nascent HLA molecules and stabilize them for expression on the cell surface. T2-A3 cells are T2 cells genetically modified to express the HLA-A*0301 allele and were a gift from Dr. Walter Storkus (University of Pittsburgh). T2-A24 cells are T2 cells genetically modified to express the HLA-A24 allele. The HLA-A24 gene was a gift from Dr. Paul Robbins (Surgery Branch, National Cancer Institute). T2 cells were grown in suspension culture in RPMI-1640 (Gibco, Grand Island, N.Y.), 10% fetal bovine serum (Hyclone, Logan, Utah) supplemented with 200μM L-Glutamine (JRH Biosciences, Lenexa, Kans.), 50 units-μg/ml Pen/Strep (Sigma, St. Louis, Mo.), 1% NEAA (Sigma, St. Louis, MO), and 1% Na-Pyruvate (Sigma, St. Louis, Mo.) in 5% CO₂ at 37° C.

Materials and Methods. Peptide/MHC binding Assays. T2 cells expressing the HLA molecule of interest were resuspended in AimV serum free media (Gibco, Grand Island, N.Y.) to a concentration of 1×10⁶ cells/ml and pulsed with 3 μg/ml beta-2 microglobulin (β₂-M) (Sigma, St. Louis, Mo.) plus peptide at 0-225 μg/ml of peptide at room temperature overnight. The cells were washed and resuspended at 2>10⁵ cells/ml. The level of stabilized MHC on the cell surface of the T2 and T2-A24 cells were analyzed by direct staining of cell samples with unlabeled anti-class I mAb W6/32 and a FITC-labeled goat-anti-mouse IgG2a secondary antibody. The level of stabilized MHC on the cell surface of the T2-A3 cells was analyzed by direct staining of cell samples with unlabeled anti-HLA-A3 mAb GAPA3 and a FITC-labeled goat-anti-mouse IgG2a secondary antibody. Viable cells, as determined by exclusion of propidium iodide (PI), were analyzed by flow cytometry on a dual laser FACS-Calibur (Becton Dickenson, San Jose, Calif.) using Cell Quest analysis software (Becton Dickenson, San Jose, Calif.).

### Example 2

To determine if PSCA was recognized by CD8+ T cells, antigen-pulsed T2 cells were screened with CD8+ T cell enriched PBL from patients that have received an allogeneic GM-CSF secreting pancreatic tumor vaccine (Jaffee et al., J. of Clinical Oncology, 19:145-156 (2001). The patients treated on the reported vaccine study received an initial vaccination 8-10 weeks following pancreaticoduodenectomy and 4 weeks prior to receiving a six month course of adjuvant chemoradiation. Six of these patients remained disease-free at the end of the six months and received up to 3 more vaccinations given one month apart. The association of in vivo post-vaccination delayed type hypersensitivity (DTH) responses to autologous tumor in three of eight patients receiving the highest two doses of vaccine was previously reported. These “DTH responders” (each of whom had poor prognostic indicators at the time of primary surgical resection) are the only patients who remain clinically free of pancreatic cancer >4 years after diagnosis. PBL obtained prior to vaccination and 28 days after the first vaccination were initially analyzed.

T2-A3 cells pulsed with the two A3 binding epitopes were incubated overnight with CD8+ T cell enriched lymphocytes isolated from the peripheral blood of patient non-DTH responder who relapsed 9 months after diagnosis) and 13 (DTH responder who remains disease-free) and analyzed using a gamma interferon (IFN-.gamma.) ELISPOT assay. The ELISPOT assay was chosen because it requires relatively few lymphocytes, is among the most sensitive in vitro assays for quantitating antigen-specific T cells, and correlates number of antigen-specific T cells with function (cytokine expression).

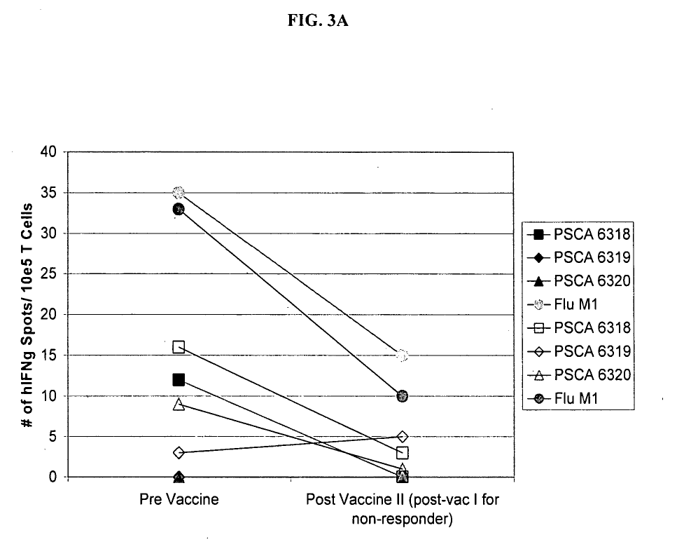

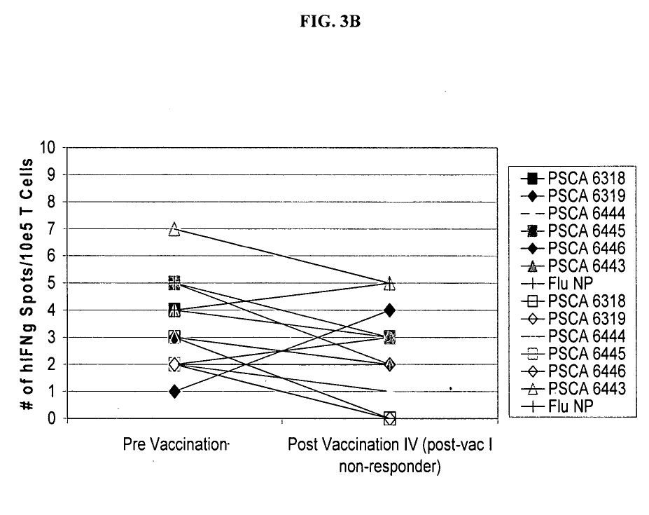

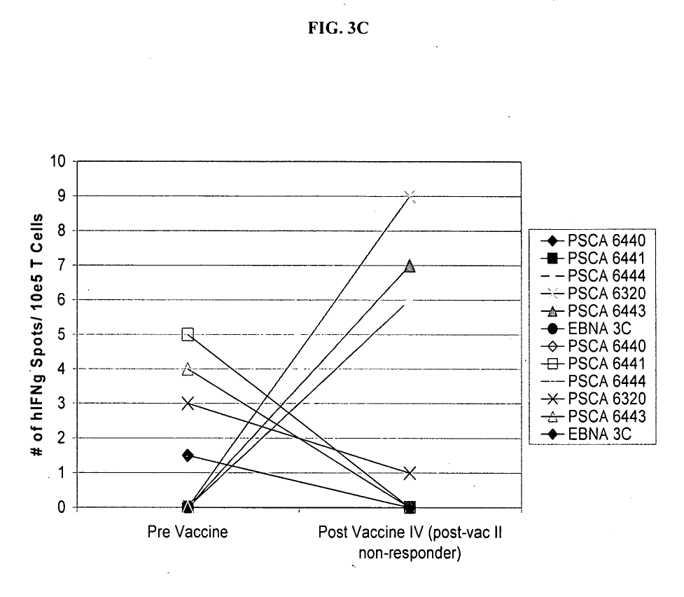

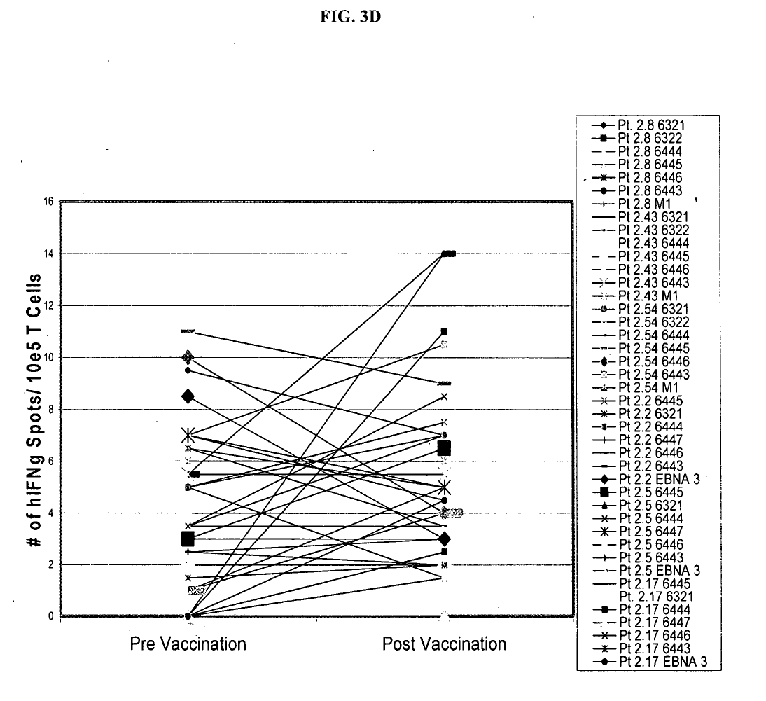

Lymphocytes from 14 patients were evaluated for the post-vaccination induction of CD8+ T lymphocytes directed against PSCA. The results indicated that PSCA did not elicit an immune response in the 3 DTH responders post-vaccination at an early stage in the study. T2 binding assays were performed with the top two ranking epitopes for HLA-A2, HLA-A3, and HLA-A24 favored by both algorithms and analyzed by ELISPOT. No post-vaccination induction of PSCA-specific T cells in any of the patients was observed; therefore, four additional PSCA peptides were synthesized for each HLA class I molecule. Analysis of these peptides also failed to demonstrate a post-vaccination induction of PSCA-specific CD8+ T cell responses (FIGS. 3A, 3B, and 3C, respectively). PSCA specific responses also could not be demonstrated in the eight non-responders (FIG. 3D).

Materials and Methods. Peripheral blood lymphocytes (PBL) and donors. Peripheral blood (100 cc pre-vaccination and 28 days after each vaccination) were obtained from all fourteen patients who received an allogeneic GM-CSF secreting pancreatic tumor vaccine as part of a previously reported phase I vaccine study. 200 cc of blood was obtained annually from all patients who completed the vaccine trial and remained disease-free. Informed consent for banking lymphocytes to be used for this antigen identification study was obtained at the time of patient enrollment into the study. Pre- and post-vaccine PBL were isolated by density gradient centrifugation using Ficoll-Hypaque (Pharmacia, Uppsala, Sweden). Cells were washed twice with serum free RPMI-1640. PBL were stored frozen at −180° C. in 90% AIM-V media containing 10% DMSO.

Materials and Methods. Enrichment ofPBL for CD8+ T cells. CD8+ T cells were isolated from thawed PBL using Magnetic Cell Sorting of Human Leukocytes as per the manufacturers directions (MACS, Miltenyi Biotec, Auburn, Calif.). Cells were fluorescently stained with CD8-PE antibody (Becton Dickenson, San Jose, Calif.) to confirm that the positive population contained CD8+ T cells and analyzed by flow cytometry. This procedure consistently yielded >95% CD8+ T cell purity.

Materials and Methods. ELISPOT assay. Multiscreen ninety-six well filtration plates (Millipore, Bedford, Mass.) were coated overnight at 4° C with 60 μl/well of 10 μg/ml anti-hIFN-γ mouse monoclonal antibody (Mab) 1-D1K (Mabtech, Nacka, Sweden). Wells were then washed 3 times each with 1×PBS and blocked for 2 hours with T cell media. Following blocking, wells were loaded with 1×10⁵ T2 cells pulsed with peptide (10 ng/ml) and 1×10⁵ freshly thawed and enriched CD8+ PBL in 200 μl T cell media in replicates of three-six. The plates were then incubated overnight at 37° C. in 5% CO₂. Cells were removed from the ELISPOT plates by washing six times with PBS+0.05% Tween 20 (Sigma, St. Louis, Mo.). Wells were incubated for 2 hours at 37° C. in 5% CO₂ using 60 μl/well of 2 μg/ml biotinylated Mab anti-hIFNgamma 7-B6-1 (Mabtech, Nacka, Sweden). After washing six times with PBS/Tween 0.05%, avidin peroxidase complex (Vectastain ELITE ABC kit, Vector Laboratories, Burlingame, Calif.) was added at 100 μl per well and incubated for one hour at room temperature. Following three washes with PBS/Tween 0.05% and three washes with PBS, AEC-substrate solution (3-amino-9-ethylcarbazole) was added at 100 μl/well and incubated for 4-12 minutes at room temperature. Color development was stopped by washing with tap water. Plates were dried overnight at room temperature and colored spots were counted using an automated image system ELISPOT reader (Axioplan2, Carl Zeiss Microimaging Inc., Thornwood, N.Y.).

### Example 3

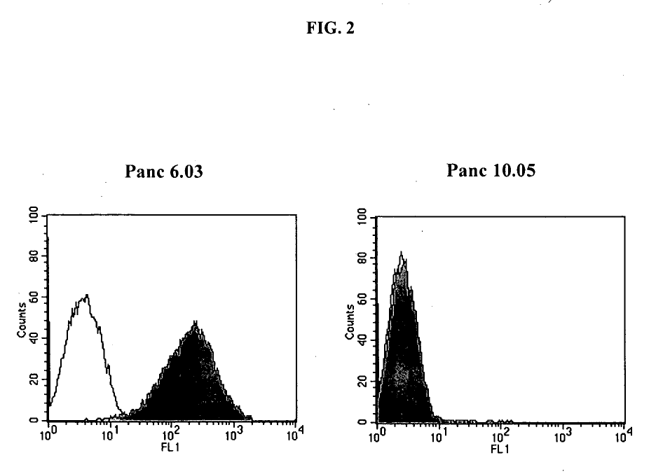

Flow cytometry analysis of PSCA expression by the two allogeneic vaccine cell lines used in the study was performed. The results are shown in FIG. 2. PSCA was found to only be expressed by one of the vaccine cell lines (Panc 6.03). The cell line, Panc 10.05, that didn't express PSCA had been cell line used in the first vaccination given to the patients in the study.

Materials and Methods. Flow cylometry. The expression of PSCA on the vaccine lines was evaluated by flow cytometric analysis. The vaccine lines were washed twice and resuspended in “FACS” buffer (HBSS supplemented with 1% PBS, 2% FBS, and 0.2% sodium azide), then stained with a PSCA-specific mouse monoclonal IgGI antibody (clone 1G8) (gift from Dr. Robert E. Reiter, UCLA) followed by FITC-labeled goat anti-mouse IgGI (BD PharMingen, San Jose, Calif.). Stained samples were analyzed using a FACS-Calibur flow cytometer (Becton Dickenson, San Jose, Calif.) and Cell Quest analysis software (Becton Dickenson, San Jose, Calif.).

### Example 4

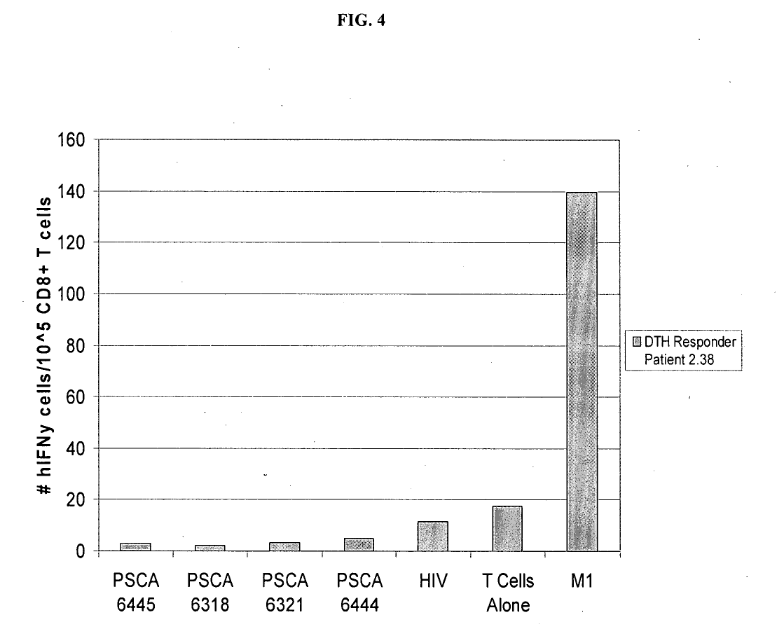

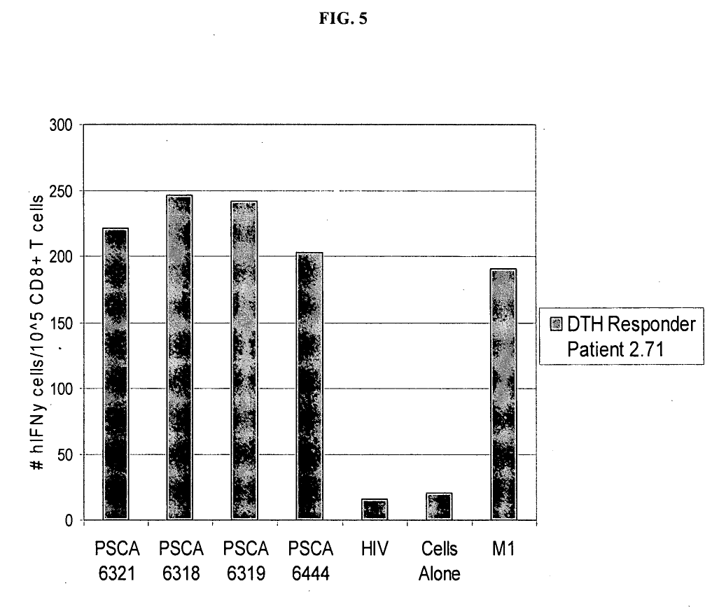

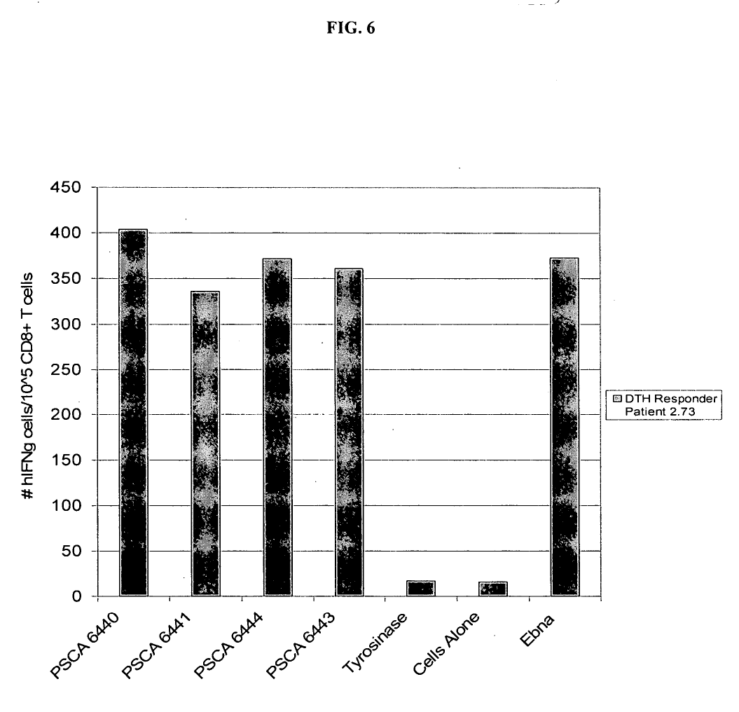

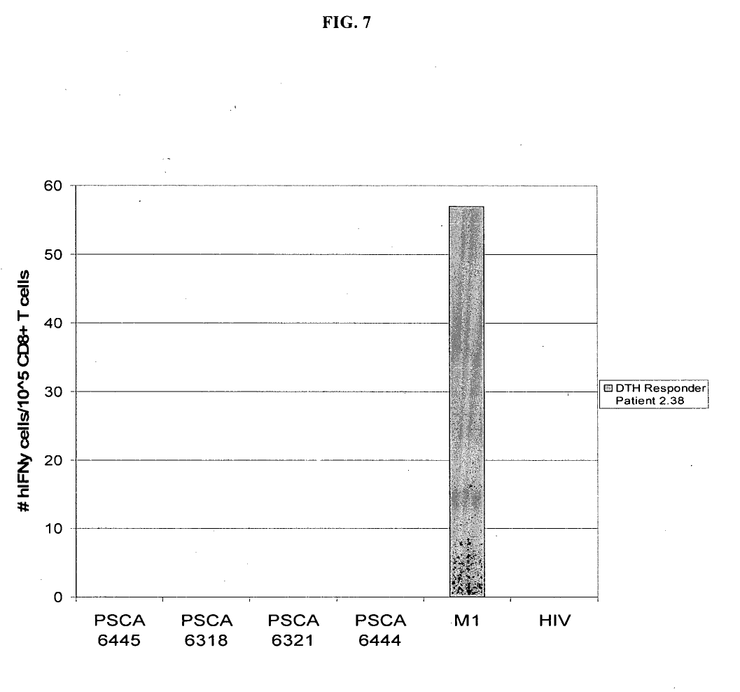

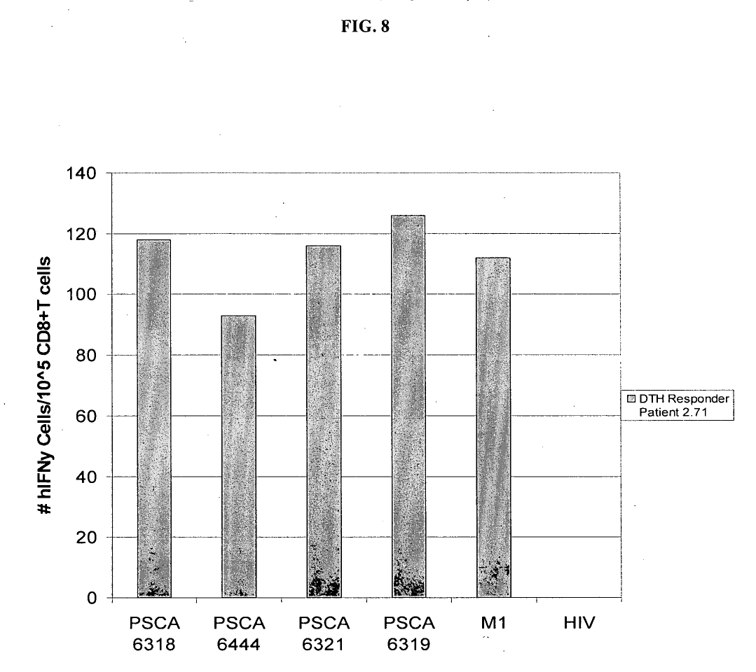

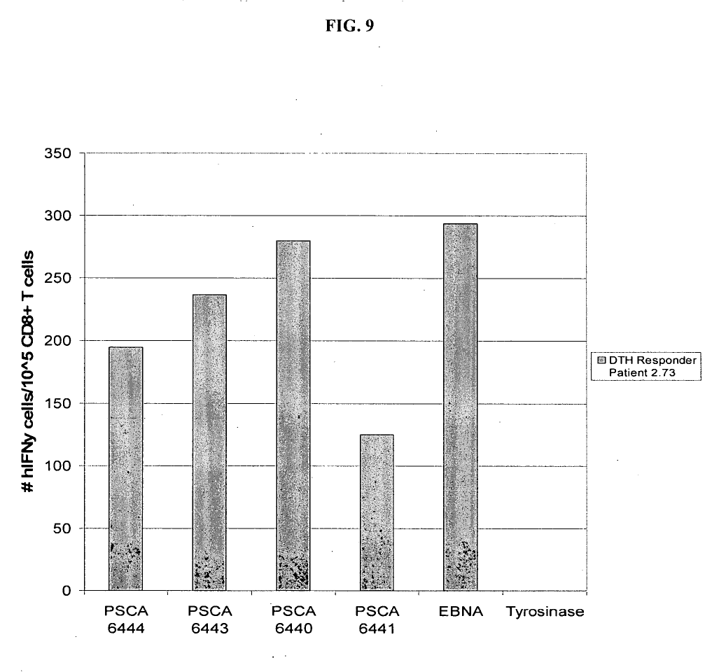

In marked contrast to the negative results seen at earlier post-vaccination time points, ELISPOT studies at a much later time point post-fourth vaccination demonstrated that two of the three DTH responders did, in fact, develop significant PSCA-specific T cell responses. HLA binding peptides corresponding to HLA alleles expressed by the treated patients (A2, A3 and A24) were synthesized and utilized in a quantitative ELISPOT assay. It was found that multiple HLA A2 binding peptides as well two HLA A3 and two HLA 24 binding peptides from PSCA were, in fact, recognized by T cells from 2 of the three vaccinated pancreatic cancer patients expressing the appropriately matched HLA alleles at a time point four years post completion of treatment. Specifically, in 2 of 3 patients demonstrating a clinical response to the pancreatic cancer vaccine, there was an increase in T cell precursor frequency to the appropriate HLA PSCA peptide of greater than five-fold post vaccination. The results of the ELISPOT experiments are shown in FIGS. 4-9. In contrast, patients receiving comparable doses of vaccine but who did not demonstrate clinical responses failed to demonstrate a significant increase in frequency of T cells responding to PSCA post vaccine. Therefore, there was a good correlation between clinical response to the genetically modified whole cell vaccine and a vaccine induced increase in T cell responses to PSCA as measured with the quantitative ELISPOT assay. (The third DTH responder who did not demonstrate a significant PSCA specific T cell response only received two vaccines (only one of which comprised a PSCA-expressing tumor cell) during the study because she subsequently developed a late autoimmune antibody mediated complication attributed to the Mitomycin-C that required medical intervention and withdrawal from the vaccine study.)

It is understood that the examples and embodiments described herein are for illustrative purposes only and that various modifications or changes in light thereof will be suggested to persons skilled in the art and are to be included within the spirit and purview of this application.

All publications, patents, patent applications, and accession numbers (including both polynucleotide and polypeptide sequences) cited herein are hereby incorporated by reference herein in their entirety for all purposes to the same extent as if each individual publication, patent, patent application or accession number were specifically and individually indicated to be so incorporated by reference.

What is claimed is:

1. A method of inducing a T-cell response to a tumor that expresses prostate stem cell antigen (PSCA), said method comprising
administering to a mammal who has said tumor or who has had said tumor removed, a composition comprising a polypeptide comprising an MHC class I-binding epitope of PSCA, whereby a T-cell response to PSCA is induced in the mammal, wherein the composition does not comprise a whole tumor cell.

2. The method of claim 1, wherein the tumor overexpresses prostate stem cell antigen relative to a normal tissue from which the tumor is derived.

3. The method of claim 1, wherein the tumor is a pancreatic cancer, a bladder cancer or a prostate cancer.

4. The method of claim 1, wherein the mammal is a human and the PSCA is human PSCA.

5. The method of claim 1, wherein the MHC class I-binding epitope is an HLA-A2-restricted epitope or an HLA-A24-restricted epitope.

6. The method of claim 1, wherein the polypeptide further comprises an MHC class II-binding epitope of PSCA.

7. The method of claim 1, wherein the polypeptide comprises a plurality of MHC class I-binding epitopes of PSCA.

8. The method of claim 1, wherein the polypeptide comprises PSCA.

9. The method of claim 1, wherein the polypeptide comprises a fragment of PSCA, wherein the fragment comprises the MHC class I epitope and is at least 8 amino acids in length.

10. The method of claim 1, wherein the polypeptide comprises a fusion protein, wherein the fusion protein comprises a first and a second portion, wherein the first portion comprises the MHC class I-binding epitope, and wherein the second portion comprises a sequence of at least 6 amino acid residues, wherein the sequence of said second portion is not in PSCA.

11. The method of claim 1, wherein the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

12. The method of claim 1, wherein the T-cell response comprises induction of PSCA-specific CD8+ T cells.

13. The method of claim 1, wherein the composition further comprises an adjuvant or a non-PSCA antigen.

14. The method of claim 1, wherein the composition is administered in an amount sufficient to induce tumor regression.

15. The method of claim 1, wherein the composition is administered in an amount sufficient to inhibit progression of a cancer in the mammal.

16. The method of claim 1, wherein the composition is administered in an amount sufficient to delay or prevent recurrence of cancer in the mammal, wherein the mammal has had said tumor removed.

17. The method of claim 1, wherein the composition comprises a recombinant vector comprising a bacterium, virus or yeast expressing the polypeptide.

18. The method of claim 17, wherein the vector comprises a bacterium and the bacterium is selected from the group consisting of Shigella flexneri, E. coli, Listeria monocytogenes, Yersinia enterocolitica, Salmonella Typhimurium, Salmonella typhi, and mycobacterium.

19. A method of inducing a T-cell response to a tumor that expresses prostate stem cell antigen (PSCA), said method comprising
administering to a mammal who has said tumor or who has had said tumor removed, a composition comprising a polynucleotide encoding a polypeptide comprising an MHC class I-binding epitope of PSCA, whereby a T-cell response to PSCA is induced in the mammal, wherein the composition does not comprise a whole tumor cell.

20. The method of claim 19, wherein the tumor overexpresses prostate stem cell antigen relative to a normal tissue from which the tumor is derived.

21. The method of claim 19, wherein the tumor is a pancreatic cancer, a bladder cancer or a prostate cancer.

22. The method of claim 19, wherein the mammal is a human and the PSCA is human PSCA.

23. The method of claim 19, wherein the MHC class I-binding epitope is an HLA-A2-restricted epitope or an HLA-A24-restricted epitope.

24. The method of claim 19, wherein the polypeptide further comprises an MHC class II-binding epitope of PSCA.

25. The method of claim 19, wherein the polypeptide comprises a plurality of MHC class I-binding epitopes of PSCA.

26. The method of claim 19, wherein the polypeptide comprises PSCA.

27. The method of claim 19, wherein the polypeptide comprises a fragment of PSCA, wherein the fragment comprises the MHC class I epitope and is at least 8 amino acids in length.

28. The method of claim 19, wherein the polypeptide comprises a fusion protein, wherein the fusion protein comprises a first and a second portion, wherein the first portion comprises the MHC class I-binding epitope of PSCA, and wherein the second portion comprises a sequence of at least 6 amino acid residues, wherein the sequence of said second portion is not in PSCA.

29. The method of claim 19, wherein the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

30. The method of claim 19, wherein the T-cell response comprises induction of PSCA-specific CD8+ T cells.

31. The method of claim 19, wherein the composition further comprises an adjuvant or a non-PSCA antigen.

32. The method of claim 19, wherein the composition is administered in an amount sufficient to induce tumor regression.

33. The method of claim 19, wherein the composition is administered in an amount sufficient to inhibit progression of a cancer in the mammal.

34. The method of claim 19, wherein the composition is administered in an amount sufficient to inhibit recurrence of cancer in the mammal, wherein the mammal has had said tumor removed.

35. The method of claim 19, wherein the composition comprises a recombinant vector comprising a bacterium, virus or yeast comprising the polynucleotide and expressing the polypeptide.

36. The method of claim 35, wherein the vector comprises a bacterium and the bacterium is selected from the group consisting of Shigella flexneri, E. coli, Listeria monocytogenes, Yersinia enterocolitica, Salmonella Typhimurium, Salmonella typhi, and mycobacterium.

37. A method of treating cancer in a mammal who has a PSCA-expressing tumor or who has had a PSCA-expressing tumor removed, comprising:
administering to the mammal a composition comprising a polypeptide comprising an MHC class I-binding epitope of PSCA or an MHC class II-binding epitope of PSCA, whereby a T-cell response to PSCA is induced in the mammal, wherein the composition does not comprise a whole tumor cell.

38. A method of treating cancer in a mammal who has a PSCA-expressing tumor or who has had a PSCA-expressing tumor removed, comprising:
administering to the mammal a composition comprising a polynucleotide encoding a polypeptide comprising an MHC class I-binding epitope of PSCA or an MHC class II-binding epitope of PSCA, whereby a T-cell response to PSCA is induced in the mammal, wherein the composition does not comprise a whole tumor cell.

39. A method of inducing a T-cell response in a mammal to a tumor that expresses prostate stem cell antigen (PSCA), said method comprising
administering to a mammal who has said tumor or who has had said tumor removed, a composition comprising a polypeptide comprising an MHC class II-binding epitope of PSCA, whereby a T-cell response to PSCA is induced in the mammal, wherein the composition does not comprise a whole tumor cell.

40. A method of inducing a T-cell response in a mammal to a tumor that expresses prostate stem cell antigen (PSCA), said method comprising
administering to a mammal who has said tumor or who has had said tumor removed, a composition comprising a polynucleotide encoding a polypeptide comprising an MHC class II-binding epitope of PSCA, whereby a T-cell response to PSCA is induced in the mammal, wherein the composition does not comprise a whole tumor cell.

41. An immunogenic composition that induces a T cell response to a PSCA-expressing tumor cell in a mammal, comprising:
a polypeptide comprising an MHC class I-binding epitope of PSCA,
wherein the immunogenic composition does not comprise a whole mammalian cell.

42. The immunogenic composition of claim 41, which comprises a recombinant vector comprising a bacterium, virus or yeast expressing the polypeptide.

43. The immunogenic composition of claim 42, wherein the vector comprises a bacterium and the bacterium is selected from the group consisting of Shigella flexneri, E. coli, Listeria monocytogenes, Yersinia enterocolitica, Salmonella Typhimurium, Salmonella typhi, and mycobacterium.

44. The immunogenic composition of claim 41, wherein the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ ID NO:10), DYYVGKKNI (SEQ ID NO:15), YYVGKKNIT (SEQ ID NO:16), and ALQPAAAIL (SEQ ID NO:9).

45. The immunogenic composition of claim 41, wherein the polypeptide further comprises an MHC class II-binding epitope.

46. The immunogenic composition of claim 41, wherein the polypeptide comprises a plurality of MHC class I-binding epitopes of PSCA.

47. The immunogenic composition of claim 41, wherein the polypeptide comprises PSCA.

48. The immunogenic composition of claim 41, wherein the polypeptide comprises a fusion protein, wherein the fusion protein comprises a first and a second portion, wherein the first portion comprises the MHC class I-binding epitope of PSCA, and wherein the second portion comprises a sequence of at least 6 amino acid residues, wherein the sequence of said second portion is not in PSCA.

49. The immunogenic composition of claim 41, wherein the T-cell response comprises induction of PSCA specific CD8+ T cells.

50. The immunogenic composition of claim 41, wherein the polypeptide comprises a fragment of PSCA, wherein the fragment comprises the MHC class I epitope and is at least 8 amino acids in length.

51. The immunogenic composition of claim 41, wherein the mammal is human.

52. An immunogenic composition that induces a T cell response to a PSCA-expressing tumor cell in a mammal, comprising:
a polynucleotide encoding a polypeptide comprising an MHC class I-binding epitope of PSCA,
wherein the immunogenic composition does not comprise a whole mammalian cell.

53. The immunogenic composition of claim 52, which comprises a recombinant vector comprising a bacterium, virus or yeast comprising the polynucleotide and expressing the polypeptide.

54. The immunogenic composition of claim 53, wherein the vector comprises a bacterium and the bacterium is selected from the group consisting of Shigella flexneri, E. coli, Listeria monocytogenes, Yersinia enterocolitica, Salmonella Typhimurium, Salmonella typhi, and mycobacterium.

55. The immunogenic composition of claim 52, wherein the MHC class I-binding epitope is selected from the group consisting of LLALLMAGL (SEQ ID NO:5), ALQPGTALL (SEQ ID NO:6), ALLMAGLAL (SEQ ID NO:8), LLPALGLLL (SEQ IDNO:10), DYYVGKKNI (SEQ ID NO:15), YYVGKKNIT (SEQ ID NO:16), and ALQPAAAIL (SEQ ID NO:9).

56. The immunogenic composition of claim 52, wherein the polypeptide further comprises an MHC class II-binding epitope.

57. The immunogenic composition of claim 52, wherein the polypeptide comprises a plurality of MHC class I-binding epitopes of PSCA.

58. The immunogenic composition of claim 52, wherein the polypeptide comprises PSCA.

59. The immunogenic composition of claim 52, wherein the polypeptide comprises a fusion protein, wherein the fusion protein comprises a first and a second portion, wherein the first portion comprises the MHC class I-binding epitope of PSCA, and wherein the second portion comprises a sequence of at least 6 amino acid residues, wherein the sequence of said second portion is not in PSCA.

60. The immunogenic composition of claim 52, wherein the T-cell response comprises induction of PSCA specific CD8+ T cells.

61. The immunogenic composition of claim 52, which further comprises an adjuvant or a non-PSCA antigen.

62. The immunogenic composition of claim 52, wherein the polypeptide comprises a fragment of PSCA, wherein the fragment comprises the MHC class I epitope and is at least 8 amino acids in length.

63. The immunogenic composition of claim 52, wherein the mammal is human.

64. A vaccine that induces a T cell response to a PSCA-expressing tumor cell in a human, comprising:
a polypeptide comprising an MHC class I-binding or MHC class II-binding epitope of PSCA; and
an adjuvant,
wherein the vaccine does not comprise a whole tumor cell.

65. A vaccine that induces a T cell response to a PSCA-expressing tumor cell in a human, comprising:
a polynucleotide encoding a polypeptide comprising an MHC class I-binding or MHC class II-binding epitope of PSCA; and
an adjuvant,
wherein the vaccine does not comprise a whole tumor cell.

66. A vaccine that induces a T cell response to a PSCA-expressing tumor cell in a human, comprising:
a whole cell from a tumor cell line that has been selected or modified to overexpress a polypeptide relative to the tumor cell line prior to selection or modification, wherein the polypeptide comprises an MHC class I-binding epitope of PSCA or an MHC class II-binding epitope of PSCA; and
an adjuvant.

67. A method of inducing in a mammal a T-cell response to a tumor that expresses prostate stem cell antigen (PSCA), said method comprising
administering to the mammal who has said tumor or who has had said tumor removed, a composition comprising a whole cell from a tumor cell line that has been selected or modified to overexpress a polypeptide relative to the tumor cell line prior to selection or modification, wherein the polypeptide comprises an MHC class I-binding epitope of PSCA or an MHC class II-binding epitope of PSCA, whereby a T-cell response to PSCA is induced in the mammal.
